(19) United States
(12) Patent Application Publication
(10) Pub. No.: US 2016/0017245 A1
(43) Pub. Date: Jan. 21, 2016

(54) MICROOGANISMS WITH ALTERED FATTY ACID PROFILES FOR RENEWABLE MATERIALS AND BIO-FUEL PRODUCTION

(71) Applicants: Paul Erik KLEIBORN, ECHT (NL); Jacob BORDEN, (US); Jon HANSEN, (US); Martin SELLERS, (US); Paul Warren BEHRENS, (US)

(72) Inventors: Kirk Emil APT, Echt (NL); Jacob BORDEN, Echt (NL); Jon Milton HANSEN, Echt (NL); Martin SELLERS, Echt (NL); Paul Warren BEHRENS, Echt (NL)

(21) Appl. No.: 14/772,408

(22) PCT Filed: Mar. 10, 2014
(86) PCT No.: PCT/US2014/022603

§ 371 Date: Sep. 3, 2015

Related U.S. Application Data
(60) Provisional application No. 61/774,717, filed on Mar. 8, 2013.

(51) Int. Cl. C10L 1/02; C12P 7/64; C12N 1/16

(52) U.S. Cl. CPC C10L 1/026; C12N 1/16; C12P 7/649; C10L 2200/0476; C10L 2270/026; C10L 2290/26

(57) ABSTRACT

Biofuel generated from the lipids of oleaginous yeast must conform to industry and regulatory standards for fuel performance and composition. In particular, precise lipid compositions and fuel properties are required for approval of biofuels. Disclosed are genetically modified microorganisms generated from oleaginous yeast that show significant alterations in lipid profile. Also disclosed are methods of producing biofuels and biofuel compositions.

### NAMES OF THE PARTIES TO A JOINT RESEARCH AGREEMENT

For purposes of 35 U.S.C. §103(c)(2), a joint research agreement was executed between BP Biofuels UK Limited and Martek Biosciences Corporation on Dec. 18, 2008 in the field of biofuels. Also for the purposes of 35 U.S.C. §103(c)(2), a joint development agreement was executed between BP Biofuels UK Limited and Martek Biosciences Corporation on Aug. 7, 2009 in the field of biofuels. Also for the purposes of 35 U.S.C. §103(c)(2), a joint development agreement was executed between BP Biofuels UK Limited and DSM Biobased Products and Services B.V. on Sep. 1, 2012 in the field of biofuels

### TECHNICAL FIELD

This application is directed to microorganisms, media, biological oils, biofuels, and/or methods suitable for use in lipid production.

### BACKGROUND

Biofuels utilized in the United States, European Union, and other parts of the world must meet government and/or industry standards to be approved for use. These standards require the extracted fatty acid methyl esters, or FAME, to contain restricted quantities of certain fatty acid (FA) components.

In general, many of the required standards are based on the FAME profile found in vegetable oil extracted from the rapeseed plant. Production of oils from microorganisms has many advantages over production of oils from plants. Microorganisms have a significantly shorter life cycle, less labor requirement, growth that is independent of season and climate, and easier scale-up. Cultivation of microorganisms also does not require large acreages and there is no competition with food production.

Many known oleaginous microorganisms that are otherwise suited for biofuel production fail to produce a FAME profile which meets accepted standards for biofuel production. Therefore FAME produced by these microorganisms must undergo time-consuming and costly distillation and/or chemical processing to modify the FAME profile to meet accepted standards. To date, no known oleaginous microorganism produces a FAME profile that meets all of the accepted standards for biofuel production. There is a need for new microorganisms that produce desirable FAME profiles that either meet or minimally deviate from the FAME and FAME-influenced specifications in the biofuels standards.

### DETAILED DESCRIPTION OF EMBODIMENTS

Biofuel, or biodiesel, is typically comprised of simple monoalkyl esters of fatty acids, or FAME, derived from transesterified oils or animal fats. It represents an attractive alternative to conventional diesel fuel, as it is made from renewable sources. However, biofuel is still faced with technical challenges, such as oxidative stability, low-temperature performance, and nitrogen oxide emissions.

Disclosed herein are novel oleaginous modified microorganisms, abbreviated OMM, suitable for biofuel production. These OMM are also suitable for production of other renewable materials. These OMM comprise a genetic modification not present in an unmodified microorganism, which in certain embodiments, alters the FAME profile of the OMM. In certain embodiments, the OMM comprises a fatty acid methyl ester (FAME) profile that differs from the FAME profile of the unmodified microorganism when grown in culture. In certain other embodiments, the genetic modification alters the fuel properties of the biofuel produced by the OMM, such as for example cold flow properties.

The disclosure also relates to methods of cultivating such microorganisms for the production of useful compounds, including lipids, fatty acid esters, fatty acids, aldehydes, alcohols, alkanes, fuels, fuel and precursors, for use in industry and fuels, or as an energy and food sources. The microorganisms as disclosed in the application can be selected or genetically engineered for use in the methods or other aspects of the according to the disclosure described herein.

### 1. DEFINITIONS

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al., eds., Springer Verlag (1991); Hale & Marham, The Harper Collins Dictionary of Biology (1991); Sambrook et al., Molecular Cloning: A Laboratory Manual, (3d edition, 2001, Cold Spring Harbor Press).

As used herein, the following terms have the meanings ascribed to them unless specified otherwise.

As used herein, the terms “has,” “having,” “comprising,” “with,” “containing,” and “including” are open and inclusive expressions. Alternately, the term “consisting” is a closed and exclusive expression. Should any ambiguity exist in construing any term in the claims or the specification, the intent of the drafter is toward open and inclusive expressions.

As used herein, the term “and/or the like” provides support for any and all individual and combinations of items and/or members in a list, as well as support for equivalents of individual and combinations of items and/or members.

Regarding an order, number, sequence, omission, and/or limit of repetition for steps in a method or process, the drafter intends no implied order, number, sequence, omission, and/or limit of repetition for the steps to the scope of the invention, unless explicitly provided.

Regarding ranges, ranges are to be construed as including all points between upper values and lower values, such as to provide support for all possible ranges contained between the upper values and the lower values including ranges with no upper bound and/or lower bound.

Basis for operations, percentages, and procedures can be on any suitable basis, such as a mass basis, a volume basis, a mole basis, and/or the like. If a basis is not specified, a mass basis or other appropriate basis should be used.

The term “substantially,” as used herein, refers to being largely that which is specified and/or identified.

The term “similar,” as used herein, refers to having characteristics in common, such as not dramatically different.

It will be apparent to those skilled in the art that various modifications and variations can be made in the disclosed structures and methods without departing from the scope or spirit of the invention. Particularly, descriptions of any of the embodiments can be freely combined with descriptions of other embodiments to result in combinations and/or variations of two or more elements and/or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

The terms “producing” and “production,” as used herein, refer to making, forming, creating, shaping, bringing about, bringing into existence, manufacturing, growing, synthesizing, and/or the like. According to some embodiments, producing includes fermentation, cell culturing, and/or the like. Producing can include new or additional organisms as well as maturation of existing organisms.

The term “growing,” as used herein, refers to increasing in size, such as by assimilation of material into the living organism and/or the like.

The term “biological,” as used herein, refers to life systems, living processes, organisms that are alive, and/or the like. Biological can refer to organisms from archaea, bacteria, and/or eukarya. Biological can also refer to derived and/or modified compounds and/or materials from biological organisms. According to some embodiments, biological excludes fossilized and/or ancient materials, such as those whose life ended at least about 1,000 years ago.

The term “oil,” as used herein, refers to hydrocarbon substances and/or materials that are at least somewhat hydrophobic and/or water repelling. Oil can include mineral oil, organic oil, synthetic oil, essential oil, and/or the like. Mineral oil refers to petroleum and/or related substances derived at least in part from the Earth and/or underground, such as fossil fuels. “Organic oil” refers to materials and/or substances derived at least in part from plants, animals, other organisms, and/or the like. “Synthetic oil” refers to materials and/or substances derived at least in part from chemical reactions and/or processes, such as can be used in lubricating oil. Oil can be at least generally soluble in nonpolar solvents and other hydrocarbons, but at least generally insoluble in water and/or aqueous solutions. Oil can be at least about 50 percent soluble in nonpolar solvents, at least about 75 percent soluble in nonpolar solvents, at least about 90 percent soluble in nonpolar solvents, completely soluble in nonpolar solvents, about 50 percent soluble in nonpolar solvents to about 100 percent soluble in nonpolar solvents and/or the like, all on a mass basis.

The term “biological oils,” as used herein, refers to hydrocarbon materials and/or substances derived at least in part from living organisms, such as animals, plants, fungi, yeasts, algae, microalgae, bacteria, and/or the like. According to some embodiments, biological oils can be suitable for use as and/or conversion into biofuels and/or renewable materials. These renewable materials can be used in the manufacture of a food, dietary supplement, cosmetic, or pharmaceutical composition for a non-human animal or human.

The term “lipid,” as used herein, refers to oils, fats, waxes, greases, cholesterol, glycerides, steroids, phosphatides, cerebrosides, fatty acids, fatty acid related compounds, derived compounds, other oily substances, and/or the like. Lipids can be made in living cells and can have a relatively high carbon content and a relatively high hydrogen content with a relatively lower oxygen content. Lipids typically include a relatively high energy content, such as on a mass basis.

The term “renewable materials,” as used herein, refers to substances and/or items that have been at least partially derived from a source and/or process capable of being replaced by natural ecological cycles and/or resources. Renewable materials can include chemicals, chemical intermediates, solvents, monomers, oligomers, polymers, biofuels, biofuel intermediates, biogasoline, biogasoline blendstocks, biodiesel, green diesel, renewable diesel, biodiesel blend stocks, biodistillates, biological oils, and/or the like. In some embodiments, the renewable material can be derived from a living organism, such as plants, algae, bacteria, fungi, and/or the like.

The term “biofuel,” as used herein, refers to components and/or streams suitable for use as a fuel and/or a combustion source derived at least in part from renewable sources. The biofuel can be sustainably produced and/or have reduced and/or no net carbon emissions to the atmosphere, such as when compared to fossil fuels. According to some embodiments, renewable sources can exclude materials mined or drilled, such as from the underground. In some embodiments, renewable resources can include single cell organisms, multicell organisms, plants, fungi, bacteria, algae, cultivated crops, noncultivated crops, timber, and/or the like. Biofuels can be suitable for use as transportation fuels, such as for use in land vehicles, marine vehicles, aviation vehicles, and/or the like. Biofuels can be suitable for use in power generation, such as raising steam, exchanging energy with a suitable heat transfer media, generating syngas, generating hydrogen, making electricity, and or the like.

The term “biodiesel,” as used herein, refers to components or streams derived from renewable resources and suitable for direct use and/or blending into a diesel pool and/or a cetane supply, Suitable biodiesel molecules can include fatty acid esters, monoglycerides, diglycerides, triglycerides, lipids, fatty alcohols, alkanes, naphthas, distillate range materials, paraffinic materials, aromatic materials, aliphatic compounds (straight, branched, and/or cyclic), and/or the like. Biodiesel can be used in compression ignition engines, such as automotive diesel internal combustion engines, truck heavy duty diesel engines, and/or the like. In the alternative, the biodiesel can also be used in gas turbines, heaters, boilers, and/or the like. According to some embodiments, the biodiesel and/or biodiesel blends meet or comply with industrially accepted fuel standards, such as B20, B40, B60, B80, B99.9, B100, and/or the like.

The term “biodistillate” as used herein, refers to components or streams suitable for direct use and/or blending into aviation fuels (jet), lubricant base stocks, kerosene fuels, fuel oils, and/or the like. Biodistillate can be derived from renewable sources, and have any suitable boiling point range, such as a boiling point range of about 100 degrees Celsius to about 700 degrees Celsius, about 150 degrees Celsius to about 350 degrees Celsius, and/or the like. In certain embodiments, the biodistillate is produced from recently living plant or animal materials by a variety of processing technologies. According to one embodiment, the biodistillates can be used for fuel or power in a homogeneous charge compression ignition (HCCI) engine. HCCI engines may include a form of internal combustion with well-mixed fuel and oxidizer (typically air) compressed to the point of auto-ignition.

The term “consuming,” as used herein, refers to using up, utilizing, eating, devouring, transforming, and/or the like. According to some embodiments, consuming can include processes during and/or a part of cellular metabolism (catabolism and/or anabolism), cellular respiration (aerobic and/or anaerobic), cellular reproduction, cellular growth, fermentation, cell culturing, and/or the like.

The term “feedstock,” as used herein, refers to materials and/or substances used to supply, feed, provide for, and/or the like, such as to an organism, a machine, a process, a production plant, and/or the like. Feedstocks can include raw materials used for conversion, synthesis, and/or the like. According to some embodiments, the feedstock can include any material, compound, substance, and/or the like suitable for consumption by an organism, such as sugars, hexoses, pentoses, monosaccharides, disaccharides, trisaccharides, polyols (sugar alcohols), organic acids, starches, carbohydrates, and/or the like. According to some embodiments, the feedstock can include sucrose, glucose, fructose, xylose, glycerol, mannose, arabinose, lactose, galactose, maltose, other five carbon sugars, other six carbon sugars, other twelve carbon sugars, plant extracts containing sugars, other crude sugars, and/or the like. Feedstock can refer to one or more of the organic compounds listed above when present in the feedstock.

According to some embodiments, the feedstock can be fed into the fermentation using one or more feeds. In some embodiments, feedstock can be present in media charged to a vessel prior to inoculation. In some embodiments, feedstock can be added through one or more feed streams in addition to the media charged to the vessel.

According to some embodiments, the feedstock can include a lignocellulosic derived material, such as material derived at least in part from biomass and/or lignocellulosic sources.

The term “organic,” as used herein, refers to carbon containing compounds, such as carbohydrates, sugars, ketones, aldehydes, alcohols, lignin, cellulose, hemicellulose, pectin, other carbon containing substances, and/or the like.

The term “biomass,” as used herein, refers to plant and/or animal materials and/or substances derived at least in part from living organisms and/or recently living organisms, such as plants and/or lignocellulosic sources. Non-limiting examples of materials comprising the biomass include proteins, lipids, and polysaccharides.

The term “cell culturing,” as used herein, refers to metabolism of carbohydrates whereby a final electron donor is oxygen, such as aerobically. Cell culturing processes can use any suitable organisms, such as bacteria, fungi (including yeast), algae, and/or the like. Suitable cell culturing processes can include naturally occurring organisms and/or genetically modified organisms.

The term “fermentation,” as used herein, refers both to cell culturing and to metabolism of carbohydrates. Fermentation may be conducted aerobically, under oxygen limited conditions or anaerobically. Fermentation can include an enzyme controlled anaerobic breakdown of an energy rich compound, such as a carbohydrate to carbon dioxide and an alcohol, an organic acid, a lipid, and/or the like. In the alternative, fermentation refers to biologically controlled transformation of an inorganic or organic compound. Fermentation processes can use any suitable organisms, such as bacteria, fungi (including yeast), algae, and/or the like. Suitable fermentation processes can include naturally occurring organisms and/or genetically modified organisms.

Biological processes can include any suitable living system and/or item derived from a living system and/or a process. Biological processes can include fermentation, cell culturing, aerobic respiration, anaerobic respiration, catabolic reactions, anabolic reactions, biotransformation, saccharification, liquefaction, hydrolysis, depolymerization, polymerization, and/or the like.

The term “organism,” as used herein, refers to an at least relatively complex structure of interdependent and subordinate elements whose relations and/or properties can be largely determined by their function in the whole. The organism can include an individual designed to carry on the activities of life with organs separate in function but mutually dependent. Organisms can include a living being, such as capable of growth, reproduction, and/or the like.

The organism can include any suitable simple (mono) cell being, complex (multi) cell being, and/or the like. Organisms can include algae, fungi (including yeast), bacteria, and/or the like. The organism can include microorganisms, such as bacteria or protozoa. The organism can include one or more naturally occurring organisms, one or more genetically modified organisms, combinations of naturally occurring organisms and genetically modified organisms, and/or the like. Embodiments with combinations of multiple different organisms are within the scope of the disclosure. Any suitable combination or organism can be used, such as one or more organisms, at least about two organisms, at least about five organisms, about two organisms to about twenty organisms, and/or the like.

In one embodiment, the organism can be a single cell member of the fungal kingdom, such as a yeast, for example. Examples of oleaginous yeast that can be used include, but are not limited to the following oleaginous yeast: Candida apicola, Candida sp., Cryptococcus curvatus, Cryptococcus terricolus, Debaromyces hansenii, Endomycopsis vernalis, Geotrichum carabidarum, Geotrichum cucujoidarum, Geotrichum histendarum, Geotrichum silvicola, Geotrichum vulgare, Hyphopichia burtonii, Lipomyces lipofer, Lypomyces orentalis, Lipomyces starkeyi, Lipomyces tetrasporous, Pichia mexicana, Rodosporidium sphaerocarpum, Rhodosporidium toruloides. Rhodotorula aurantiaca, Rhodotorula dairenensis, Rhodotorula diffluens, Rhodotorula glutinus, Rhodotorula gracilis, Rhodotorula graminis, Rhodotorula minuta, Rhodotorula mucilaginosa, Rhodotorula mucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri, Trichosporon montevideense, Trichosporon pullulans, Trichosporon sp., Wickerhamomyces canadensis, Yarrowia lipolytica, and Zygoascus meyerae.

The organism can operate, function, and/or live under any suitable conditions, such as anaerobically, aerobically, photosynthetically, heterotrophically, and/or the like.

The term “oleaginous,” as used herein, refers to oil bearing, oil containing and/or producing oils, lipids, fats, and/or other oil-like substances. The oil, lipid, fat, and/or other oil-like substances may be produced inside or outside the cell. Oleaginous may include organisms that produce at least about 20 percent by weight of oils, at least about 25 percent by weight oils, at least about 30 percent by weight of oils, at least about 40 percent by weight oils, at least about 50 percent by weight oils, at least about 60 percent by weight oils, at least about 70 percent by weight oils, at least about 80 percent by weight oils, and/or the like. Oleaginous may refer to a microorganism during culturing, lipid accumulation, at harvest conditions, and/or the like.

The term “genetic engineering,” as used herein, refers to intentional manipulation and/or modification of at least a portion of a genetic code and/or expression of a genetic code of an organism.

The term “genetic modification,” as used herein, refers to any method of introducing a genetic change to an organism. Non-limiting examples include genomic mutagenesis, addition and/or removal of one or more genes, portions of proteins, promoter regions, noncoding regions, chromosomes, and/or the like. Genetic modification can be random or non-random. Genetic modification can comprise, for example, mutations, and can be insertions, deletions, point mutations, substitutions, and any other mutation. Genetic modification can also be used to refer to a genetic difference a non-wild type organism and a wild type organism.

The terms “unmodified organism” or “unmodified microorganism,” as used herein, refer to organisms, cultures, single cells, biota, and/or the like at least generally without intervening actions by exterior forces, such as humankind, machine, and/or the like. As used herein, an unmodified microorganism is typically the particular microorganism as it exists prior to introduction of a genetic modification according to this disclosure. In many embodiments, an unmodified microorganism is the wild type strain of the microorganism. However, the unmodified microorganism as defined herein can be an organism that was genetically altered previously, for example prior to the introduction of the genetic modification according to this disclosure. For example, a yeast strain available from ATCC that comprises a knockout mutation of a certain gene would be considered an unmodified microorganism according to this definition. The term unmodified microorganism also encompasses organisms that do not have a genetic modification associated with fatty acid production, FAME profile, or fuel properties.

In some embodiments, the organisms as disclosed produce fatty acids and/or contain fatty acids, such as within or on one or more vesicles and/or pockets. In the alternative, the fatty acid can be relatively uncontained within the cell and/or outside the cell, such as relatively free from constraining membranes. Producing the organism can include cellular reproduction (more cells) as well as cell growth (increasing a size and/or contents of the cell, such as by increasing a fatty acid content). Reproduction and growth can occur at least substantially simultaneously with each other, at least substantially exclusively of each other, at least partially simultaneously and at least partially exclusively, and/or the like.

Polysaccharides (also called “glycans”) are carbohydrates made up of monosaccharides joined together by glycosidic linkages. Polysaccharides are broadly defined molecules, and the definition includes intercellular polysaccharides, secreted polysaccharides, exocellular polysaccharides, cell wall polysaccharides, and the like. Cellulose is an example of a polysaccharide that makes up certain plant cell walls. Cellulose can be depolymerized by enzymes to produce monosaccharides such as xylose and glucose, as well as larger disaccharides and oligosaccharides. The quantity of each monosaccharides component following depolymerization of polysaccharides is defined herein as a monosaccharide profile. Certain polysaccharides comprise non-carbohydrate substituents, such as acetate, pyruvate, succinate, and phosphate.

The term “fatty acids,” as used herein, refer to saturated and/or unsaturated monocarboxylic acids, such as in the form of glycerides in fats and fatty oils. Glycerides can include acylglycerides, monoglycerides, diglycerides, triglycerides, and/or the like. Fatty acid also refers to carboxylic acids having straight or branched hydrocarbon groups having from about 8 to about 30 carbon atoms. The hydrocarbon groups including from 1 to about 4 sites of unsaturation, generally double or pi bonds. Examples of such fatty acids are lauric acid, steric acid, palmitic acid, oleic acid, linoleic acid, linolenic acid, arachidonic acid, elaidic acid, linoelaidicic acid, eicosenoic acid, phytanic acid, behenic acid, and adrenic acid.

Double bonds refer to two pairs of electrons shared by two atoms in a molecule.

The term “unit,” as used herein, refers to a single quantity regarded as a whole, a piece and/or complex of apparatus serving to perform one or more particular functions and/or outcomes, and/or the like.

The term “stream,” as used herein, refers to a flow and/or a supply of a substance and/or a material, such as a steady succession. Flow of streams can be continuous, discrete, intermittent, batch, semibatch, semicontinuous, and/or the like.

The term “vessel,” as used herein, refers to a container and/or holder of a substance, such as a liquid, a gas, a fermentation broth, and/or the like. Vessels can include any suitable size and/or shape, such as at least about 1 liter, at least about 1,000 liters, at least about 100,000 liters, at least about 1,000,000 liters, at least about 1,000,000,000 liters, less than about 1,000,000 liters, about 1 liter to about 1,000,000,000 liters, and/or the like. Vessels can include tanks, reactors, columns, vats, barrels, basins, and/or the like. Vessels can include any suitable auxiliary equipment, such as pumps, agitators, aeration equipment, heat exchangers, coils, jackets, pressurization systems (positive pressure and/or vacuum), control systems, and/or the like.

The term “dispose,” as used herein, refers to put in place, to put in location, to set in readiness, and/or the like. The organism can be freely incorporated into a fermentation broth (suspended), and/or fixed upon a suitable media and/or surface within at least a portion of the vessel. The organism can be generally denser than the broth (sink), generally lighter than the broth (float), generally neutrally buoyant with respect to the broth, and/or the like.

The term “adapted,” as used herein, refers to make fit for a specific use, purpose, and/or the like.

The term “meeting,” as used herein, refers to reaching, obtaining, satisfying, equaling, and/or the like.

The term “exceeding,” as used herein, refers to extending beyond, to surpassing, and/or the like. According to some embodiments, exceeding includes at least 2 percent above threshold amount and/or quantity.

Cell density (of the organism) measured in grams dry weight per liter (of the fermentation media or broth), measures and/or indicates productivity of the organism, utilization of the fermentation media (broth), and/or utilization of fermentation vessel volume. Increased cell density can result in increased production of a particular product and increased utilization of equipment (lower capital costs). Generally, increased cell density is beneficial, but too high a cell density can result in higher mixing and pumping costs (increased viscosity) and/or difficulties in removing heat (lower heat transfer coefficient), and/or the like.

The term “viscosity,” as used herein, refers to the physical property of fluids that determines the internal resistance to shear forces. Viscosity can be measured by several methods, including for example a viscometer, with typical units of centipoise (cP). Viscosity can also be measured using other known devices, such as a rheometer.

The term “density,” as used herein, refers to a mass per unit volume of a material and/or a substance. Cell density refers to a mass of cells per unit volume, such as the weight of living cells per unit volume. It is commonly expressed as grams of dry cells per liter. The cell density can be measured at any suitable point in the method, such as upon commencing fermentation, during fermentation, upon completion of fermentation, over the entire batch, and/or the like.

The term “FAME”, as used herein, refers to a fatty acid methyl ester. The term FAME may also be used to describe the assay used to determine the fatty acid quantity or percentage in a microorganism.

The term “FAME profile”, as used herein, refers to the composition of all of the individual fatty acid methyl esters that may be derived from the fatty acids produced from material made by a microorganism. This profile also represents the types and proportions of fatty acids present in the lipids of cells. In the protocol to determine FAME profile, fatty acids are commonly converted to FAME as a means to quantify the fatty acid profile of an organism. Therefore FAME profile and FA profile can be used interchangeably. In some embodiments, the term will refer to all of the fatty acids produced by a microorganism, in terms of content, composition, quantity, or percentage of total fatty acids. Typically this term will describe the mass fraction (m/m) or volume fraction (v/v) content of a particular FAME or fatty acid over the total FAME or fatty acids.

The term “desirable”, as used herein, refers generally to comprising certain properties that enhance the production of renewable materials. For example, a “desirable FAME profile” comprises certain properties that enhance the production of renewable materials. Non-limiting examples of properties that enhance the production of renewable materials are embodied in, for example, the biofuels standards and specifications, including American Society for Testing and Materials ASTM D6751 or European Committee for Standardization standard EN14214. All of the specifications and/or criteria listed in these biofuel standards and specifications documents are examples of desirable properties. Further non-limiting examples of desirable properties are the properties listed in Table 2. Any fuel property can be considered a desirable property. Cold flow properties are further non-limiting examples of these properties. For example, regarding cold flow properties, an exemplary desirable FAME profile would be one that meets the particular biofuel standard criteria for cloud point, pour point, CFPP, and/or the like, while an exemplary less desirable FAME profile would be one that fails to meet any of these properties.

In certain embodiments, the term “desirable” and/or “more desirable” is used when comparing the FAME profile of two or more organisms. In these embodiments, the more desirable FAME profile is the profile that comprises a greater number of properties that enhance the production of renewable materials. In some embodiments, the more desirable FAME profile will meet a greater number of the criteria listed in the biofuel standards and specifications than the less desirable FAME profile. Therefore the more desirable FAME profile will require less modification to meet the biofuel standards than the less desirable FAME profile. In other embodiments, the more desirable FAME profile is the profile that is more similar to the FAME profile of rapeseed.

The term “yield,” as used herein, refers to an amount and/or quantity produced and/or returned as compared to a quantity consumed. As non-limiting examples, the quantity consumed can be sugars, carbon, oxygen, or any other nutrient. “Yield” can also refer to an amount and/or quantity produced and/or returned as compared to a time period elapsed.

The term “content,” as used herein, refers to an amount of specified material contained. Dry mass basis refers to being at least substantially free from water. The fatty acid content can be measured at any suitable point in the method, such as upon commencing fermentation, during fermentation, upon completion of fermentation, over the entire batch, and/or the like.

### 2. BIOFUEL STANDARDS

The feedstock for biofuel production varies considerably with location according to climate and feedstock availability. Generally, the most abundant lipid in a particular region is the most common feedstock. In the U.S. it is most commonly produced from soybean oil. In Europe, it is most commonly produced from rapeseed oil. Animal fats or used cooking oils are also used as biofuel. Common feedstocks and their corresponding FAME profile and cold flow properties are shown in Table 1.

TABLE 1
Comparison of typical FAME (FA) profiles
of common biodiesel feedstocks and cold flow fuel
properties (CP and PP) of the corresponding FAME.

| | Oil: | | | | | |
|---|---|---|---|---|---|---|
| | | | | | Beef | Yellow |
| | Rapeseed | Sunflower | Palm | Soybean | Tallow | Grease |
| | Region: | | | | | |
| | Europe | Europe | Tropical | USA | USA | USA |
| 14:0 | tr | tr | tr | tr | 3 | 1 |
| 16:0 | 4 | 4 | 44 | 11 | 27 | 17 |
| 18:0 | 2 | 5 | 4 | 4 | 7 | 11 |
| 16:1 | tr | tr | tr | tr | 11 | 2 |
| 18:1 | 56 | 81 | 40 | 22 | 48 | 56 |
| 18:2 | 26 | 8 | 10 | 53 | 2 | 10 |
| 18:3 | 10 | tr | tr | 8 | tr | 2 |
| other | 2 | 2 | 2 | 2 | 2 | 1 |
| CP of | −3 | 0 | 16 | 0 | 17 | 8 |
| FAME | | | | | | |
| PP of | −9 | −3 | 13 | -2 | 15 | 6 |
| FAME | | | | | | |

Each region of the world has developed, or is currently developing, biofuel standards to fit prevailing regional, agricultural, and political requirements. Biofuels utilized in the United States, European Union, and other parts of the world must meet government and/or industry standards to be approved for use. In addition to other properties, these standards often require the extracted fatty acid methyl esters, or FAME, to contain restricted quantities of certain fatty acid components. Each of the parameters listed within the specifications is designed and limited to ensure that the product is fit for purpose. Biofuels must conform to the specifications to help ensure that biodiesel may be used as a fuel without causing harm. One reason for restrictions on the FAME profile is to exclude components of biodiesel with less desirable properties, for example, components that decrease oxidative stability.

While many of these specifications are related to fuel quality issues, such as completeness of the transesterification reaction or storage conditions, several parameters are influenced by the FAME profile of the biofuel. Among these specifications are cetane number, kinematic viscosity, oxidative stability, and cold-flow properties in the form of the cloud point (CP) or cold-filter plugging point (CFPP). Other important properties to consider that are influenced by fatty ester composition but are not contained in biodiesel standards are exhaust emissions, lubricity, and heat of combustion. Further specifications influenced by FAME profiles are listed in Knothe, Energy & Fuels, 22:1358-64 (2008), hereby incorporated by reference.

Many FAME standards, especially in Europe, consider the oil extracted from rapeseed as the optimal FAME profile for biofuel production.

In one aspect, the OMM described herein comprise a rapeseed-like FAME profile. In a specific embodiment, the OMM comprises a FAME profile comprising an oleic acid mass fraction (m/m) of about 50 percent to about 70 percent, a linoleic acid mass fraction (m/m) of about 15 percent to about 35 percent, and a palmitic acid mass fraction (m/m) about 1.0 percent to about 10 percent.

According to other embodiments, the OMM described herein comprise a FAME profile at least substantially similar to the FAME profile found in rapeseed. Substantially similar FAME profiles can include having a profile at least about 50 percent like rapeseed, at least about 60 percent like rapeseed, at least about 70 percent like rapeseed, at least about 80 percent like rapeseed, at least about 90 percent like rapeseed, at least about 95 percent like rapeseed, at least 99 percent like rapeseed, less than about 90 percent like rapeseed, about 50 percent like rapeseed to about 99 percent like rapeseed, and/or the like. FAME profile measurements can be, for example, FAME mass fraction and/or volume fraction of total FAME.

OMM comprising FAME profiles different from rapeseed are also within the scope of this disclosure and are described further herein. In one aspect, the OMM disclosed herein were developed, at least in part, to generate the FAME profiles according to Table 2. In some embodiments, the OMM comprise a FAME profile that satisfies one or more of the FAME range restrictions depicted in Table 2. The terms “% (m/m)” and “% (V/V)” are used to represent respectively the mass fraction and the volume fraction.

TABLE 2
Oleaginous modified microorganism target specifications: FAME profiles
and Cold Flow Fuel Properties

| | Lipid | | | | Test |
|---|---|---|---|---|---|
| Property | Numbers | Unit | Min | Max | Method |
| Appearance | | | Bright | | visual |
| | | | and | | |
| | | | clear | | |
| Myristic acid | C14:0 | % (m/m) | — | 1.5 | EN 14103 |
| Palmitic acid | C16:0 | % (m/m) | 1.0 | 10.0 | EN 14103 |
| Stearic acid | C18:0 | % (m/m) | 0.5 | 2.5 | EN 14103 |
| Arachidic acid | C20:0 | % (m/m) | — | 1.5 | EN 14103 |
| Behenic acid | C22:0 | % (m/m) | — | 1.5 | EN 14103 |
| Lignoceric acid | C24:0 | % (m/m) | — | 2.0 | EN 14103 |
| Palmitoleic acid | C16:1 | % (m/m) | — | 1.0 | EN 14103 |
| Oleic acid | C18:1 | % (m/m) | 50.0 | 70.0 | EN 14103 |
| Eicosenoic acid | C20:1 | % (m/m) | — | 3.0 | EN 14103 |
| Erucic acid | C22:1 | % (m/m) | — | 5.0 | EN 14103 |
| Linoleic acid | C18:2 | % (m/m) | 15.0 | 35.0 | EN 14103 |
| Linolenic acid | C18:3 | % (m/m) | 6.0 | 12.0 | EN 14103 |
| Water content for | | % (m/m) | — | 300 | EN ISO |
| FAME supplier | | | | | 12937 |
| CFPP Winter | — | ° C. | — | −10 | EN 116 |
| (01.10-14.04) | | | | | |
| CFPP Summer | — | ° C. | — | 0 | EN 116 |
| (15.04-30.09) | | | | | |
| Pour Point Winter | — | ° C. | −18 | −9 | EN 3016 |
| (01.10-14.04) | | | | | |
| Pour Point | — | ° C. | −18 | — | EN 3016 |
| Summer | | | | | |
| (15.04-30.09) | | | | | |
| Cloud Point | — | ° C. | — | −3 | EN 23015 |
| (01.10-14.04) | | | | | |

To date, no known oleaginous microorganism produces a FAME profile that meets all of the accepted specifications in the standards for biofuel production. For example, strains of oleaginous yeast tend to comprise FAME profiles with high levels of saturated FA, particularly for palmitic acid (16:0) and stearic acid (18:0), which are undesirable for a FAME-based biodiesel. See, e.g., Moss et al. J. Clin. Micro. 16:1073-1079 (1982); Turcotte, Adv. in Biochem. Eng. 40:74-92 (1989). The FAME produced by such microorganisms therefore requires labor-intensive distillation and/or chemical processing to bring the FAME within the accepted ranges. The more deviation from acceptable FAME ranges, the increase in cost for distillation and/or chemical processing.

In one aspect, the OMM disclosed herein comprise a desirable FAME profile. In certain embodiments, the OMM comprise a more desirable FAME profile than the unmodified microorganism. In other embodiments, the OMM comprise a more desirable FAME profile than another oleaginous organism. For example, the OMM disclosed herein may comprise a more desirable FAME profile than the FAME profile of an unrelated microorganism, such as a yeast or algae. In these embodiments, since the OMM comprises a FAME profile that meets more biofuel standards than the other organism, the OMM disclosed herein would replace the other oleaginous organism as the preferred organism for renewable material production.

The OMM disclosed herein produce FAME that require reduced distillation and/or chemical processing for meeting the specifications of the biofuel standards. In certain embodiments, the OMM comprise FAME profiles satisfying one or more of the disclosed specifications for biofuel production. While recognizing the uncertainty of evolving biofuel standards, some OMM embodiments disclosed herein will produce FAME profiles satisfying one or more future FAME standards. In other embodiments, the OMM comprise desirable FAME profiles according to future biofuel standards.

In one aspect, the OMM FAME profiles disclosed herein can meet and/or exceed particular international standards EN14214:2008, Automotive fuels, Fatty acid methyl esters (FAME) for diesel engines, November 2008 and/or ASTM D6751-09, Standard Specification for Biodiesel Fuel Blend Stock (B100) for Middle Distillate Fuels. For all published standards documents, the number immediately following the designation indicates the year of original adoption or, in the case of revision, the year of last revision. The entire contents of ASTM D6751-09 and EN14214:2008, including the country-specific versions of EN14214 and the references cited therein, are hereby incorporated by reference in their entirety as a part of this specification.

The common international standard for biofuels is published in EN14214:2008. The European Union Standard specifies several relevant characteristics, requirements, and test methods for marketed and delivered FAME to be used either as biodiesel fuel at 100 percent concentration (denoted “B100”), or as an extender for automotive fuel for diesel engines. EN14214 provides restrictions on the FA profile to exclude components of biodiesel with undesirable properties. The national standards organizations of the following countries are bound to implement this European Standard EN14214: Austria, Belgium, Czech Republic, Denmark, Finland, France, Germany, Greece, Hungary, Iceland, Ireland, Italy, Luxembourg, Malta, Netherlands, Norway, Portugal, Slovakia, Spain, Sweden, Switzerland and the United Kingdom. However, EU member states also revise and adopt their own versions of the EU fuel standards. For example, fuel refineries and terminals in Germany must comply with DIN EN14214, and those in the United Kingdom must comply with BS EN14214, both of which are incorporated by reference.

To satisfy EN14214, FAME profiles must be established using regulated testing methods. For example, EN14103:2011, Fat and oil derivatives, Fatty Acid Methyl Esters (FAME), Determination of ester and linolenic acid methyl ester contents, April 2011, must be used for determining ester content in EN14214. EN14103 is a gas chromatographic (GC) method utilizing a 30-m CARBOWAX (or comparable) column for determining FAME profile, and is hereby incorporated by reference in its entirety. In practice, these rigorous standards serve to limit the microorganisms that are suitable for biofuel production, as well as excluding certain feedstocks.

In one embodiment, the OMM disclosed herein comprise a FAME profile satisfying one or more FAME standards of the European Union. In a particular embodiment, the OMM disclosed herein comprise a FAME profile satisfying all of the FAME standards of the European Union. In another embodiment, the OMM disclosed herein comprise a FAME profile satisfying one or more FAME standards described in document EN14214:2008, as well as the standards that revise or supersede EN14214:2008.

In the United States and Canada, the biofuel standards are described in the ASTM D6751:2008 standard series (Standard Specification for Biodiesel Fuel Blend Stock (B100) for Middle Distillate Fuels, November 2008). ASTM D6751 shares several requirements with EN14214, but does not explicitly limit components of FAME profiles. However, several of the restricted properties in ASTM D6751 are influenced and/or dependent on FAME profiles. Biofuel properties are influenced by the number of carbons in the fatty acid chains, the degree of saturation of the fatty acid chains, and the alcohol to which the fatty acid chains are esterified. Residual constituents from biofuel raw materials and production processes can affect fuel filter operation with biofuel and biofuel blends as fuel temperatures become colder, as can contaminants that accumulate during fuel storage and distribution.

This disclosure is intended to cover FAME profiles that satisfy such FAME-influenced specifications as listed in the biofuel standards. In certain embodiments, the OMM comprise FAME profiles that satisfy specifications in biofuel standards that are affected or influenced by FAME profiles. Example specifications that are influenced by FAME profile include cetane number, kinematic viscosity, cloud point, cold-filter plugging point (CFPP), and oxidative stability. More detail on FAME influence on these and other biofuel standard specifications is described in Knothe, Energy & Fuels, 22:1358-1364 (2008), which is herein incorporated by reference in its entirety. Other non-limiting examples of properties of a biofuel that are determined by the FAME profile include ignition quality, heat of combustion, cold flow, oxidative stability, viscosity and lubricity.

Compliant biofuels conform to the detailed requirements listed in ASTM D6751. Basic industrial tests to determine whether the products conform to these standards typically include gas chromatography (GC), HPLC, and others. In some embodiments, produced biofuels meeting these quality standards is very non-toxic, with a toxicity rating of greater than 50 mL/kg.

In one embodiment, the OMM disclosed herein comprise a FAME profile satisfying one or more of the specifications in the standards of the United States and/or Canada. In another embodiment, the OMM disclosed herein comprise a FAME profile satisfying one or more specifications described in document ASTM D6751:2008.

In yet another embodiment, the OMM disclosed herein comprise a FAME profile satisfying one or more specifications described in either document EN14214:2008 or ASTM D6751:2008.

In another embodiment, the OMM disclosed herein comprise a FAME profile satisfying one or more specifications of any country. In another embodiment, the OMM disclosed herein comprise a FAME profile satisfying one or more specifications of the European Union, the United States, and/or Canada.

### 3. FATTY ACID METHYL ESTERS (FAME)

The oleaginous microorganisms as disclosed herein produce fatty acids (FA). The basic structure of fatty acids is a hydrophobic polycarbon chain which can vary in chain length. The FA with less than 6 carbon chains are typically known as short chain FA. The FA with less than 14 carbon chains are typically known as medium chain FA. The FA with 14 or more carbon chains are typically known as long chain FA. FA are also categorized by degrees of saturation. FA are typically known as “saturated” if they comprise no carbon double bonds, and “unsaturated” if they comprise one or more carbon double bonds.

Raw (unesterified) oils are unsuitable for biofuel use. Therefore, the raw oils produced by the organisms undergo transesterification, producing FAMEs, which are more suitable than fats or fatty acids for use as a biofuel. The resulting FAME possess fuel and physical properties, such as cold flow properties, that are competitive with petrodiesel.

A FAME can be created by an alkali catalyzed reaction between fats or fatty acids and methanol, to produce a fuel or assay a FAME profile produced by a microorganism. The esterification reaction involves the condensation of the carboxyl group of an acid and the hydroxyl group of an alcohol. For example, in rapeseed oil, fatty acids are esterified with the trivalent alcohol glycerine, the glycerine molecule is linked to three long fatty acid chains. In a simple chemical reaction in a re-esterification plant the three fatty acids change places on the trivalent glycerine with monovalent methanol in the presence of a catalyst. In this way three individual FAME molecules and one glycerine molecule are produced. For a more detailed protocol, see e.g. Moser et al., Eur. J. Lipid Sci. Technol. 109:17-24 (2007), which is hereby incorporated by reference in its entirety.

Transesterification can include use of any suitable alcohol, such as methanol, ethanol, propanol, butanol, and/or the like. Esterification can be done in the presence of a catalyst (such as boron trichloride). The catalyst protonates an oxygen atom of the carboxyl group, making the acid much more reactive. An alcohol then combines with the protonated acid to produce an ester with the loss of water. The catalyst is removed with the water. The alcohol that is used determines the alkyl chain length of the resulting esters, with the use of methanol will result in the formation of methyl esters whereas the use of ethanol will result in ethyl esters.

The types and proportions of fatty acids present in the lipids of cells, also known as the FAME profile, are major phenotypic traits and can be used to identify microorganisms. FAME offer excellent stability, and provide quick and quantitative samples for gas chromatograph (“GC”) analysis. In certain embodiments, the FAME profile is determined using gas chromatography. The methods of GC are well known in the art, as described in Freedman, B., et al., J. Am. Oil Chem. Soc. 63:1370-1375 (1986), hereby incorporated by reference. GC analysis using can determine the lengths, bonds, rings and branches of the FAME.

For example, the standard reference method for determining FAME according to both EN14214 is EN14103, a gas chromatography (GC) method utilizing a 30-M Carbowax (or comparable) column for determining FAME profile. The purpose of EN14103 is to describe a procedure for the determination of the ester content in FAME intended for incorporation into diesel oil. It also allows determining the content of specific FAMEs, such as linolenic acid methyl ester. It allows verifying that the total ester content is greater than 90.0% (m/m) and that the linolenic acid content is between 1.0% (m/m) and 15.0% (m/m).

The EN14103 method is suitable for FAME which contains methyl esters between C6 and C24. The GC temperature program of EN14103 requires modification for FAME profiles containing shorter-chain FAME because otherwise erroneous results are obtained for these species. See Schober, S., et al., Eur. J. Lipid Sci. Technol. 108:309-314 (2006), hereby incorporated by reference. Several of these modifications have been successfully implemented and are available in the published literature. For example, some modified EN14103 methods of analysis use a FAME mixture at known concentration rather than a single standard, and/or determining the response and retention time of each component experimentally. The disclosure herein is not intended to be limited to any particular methodology for determining FAME profiles.

The method of EN14103 can also be used to determine other properties of the FAME illustrated in any other biofuel standards, including the standards required by ASTM in the United States. See, e.g. Knothe, J. Am. Oil Chem. Soc., 83(10):823-832 (2006), hereby incorporated by reference. In certain embodiments, the FAME profile of an organism is determined in order to determine one or more of the biofuel standards that are affected by FAME profile.

In biofuel production, certain FA are desirable, while others are not desirable. The production standards reflect this, and the FAME must be in accordance with the limits specified in the standards. Desirable FA must be produced and/or present at a certain level. Undesirable FA should be limited to low production levels. And yet other FA should be present in a narrow range of production levels.

Many microorganisms that initially appear suited for biofuel production actually turn out not to be, and one large reason is their production of an undesirable FA profile. Oleaginous yeast described in the literature, for example, to date have generally high levels of saturated fatty acids, or “saturates.” In particular, 16:0 and 18:0, which are undesirable for a FAME based biodiesel. By using microorganisms that are predisposed to produce FAME profiles within specification levels, biofuel producers can avoid the cost and time of purifying the FAME to meet biofuel standards. These FAME specifications represent the FAME profile targets for the OMM disclosed herein.

A high level of total saturates is particularly problematic in biofuel production since it cannot easily be reduced by physical back-end refining of the FAME. As noted, many strains of oleaginous yeast generally comprise high levels of saturates which render them unsuitable for producing FAME based biodiesel.

In certain embodiments, the OMM disclosed herein comprise a FAME profile comprising a lower mass fraction (m/m) of saturated fatty acids than the FAME profile of the unmodified microorganism when grown in culture. In other embodiments, the OMM comprises a lower mass fraction (m/m) of total saturates than the unmodified microorganism when grown in culture. In other embodiments, the FAME profile of the OMM comprises a saturated fatty acid mass fraction (m/m) that is at least about 30 percent less than the FAME profile of the unmodified microorganism when grown in culture. In certain embodiments, the OMM disclosed herein comprise a FAME profile comprising a mass fraction (m/m) of saturated fatty acids lower than about 19, 10, 5, or 3 percent when grown in culture.

Rapeseed oil FAME comprises a mass fraction of total saturates of about 7.6 percent. In one embodiment, the FAME profile of the OMM comprise a mass fraction of total saturates that is substantially similar to the FAME profile of rapeseed oil. In other embodiments, the OMM disclosed herein comprise a FAME profile comprising a saturated fatty acid mass fraction (m/m) between about 7 percent and about 10 percent when grown in culture. In further embodiment, the OMM comprises a total saturates mass fraction (m/m) of about 7.6 percent when grown in culture.

In other embodiments, the FAME profile of the OMM comprises a total saturates mass fraction (m/m) of about 19 percent or less when grown in culture. OMM may also comprise a total fat mass fraction (m/m) of about 50 percent or greater in some disclosed embodiments.

Of the saturated FAME, long chain saturates are particularly disfavored for the production of biofuels. Presence of long chain saturate fatty esters can produce direct or indirect negative effects on cold properties of biofuels. For example, cloud point in particular is increased by the presence of long chain saturates, and saturated fatty acids of chain lengths greater than C12 have shown to increase the PP substantially. Further, it has been shown that the cetane number, a dimensionless descriptor related to the ignition quality of a diesel fuel, decreases with a decreasing chain length, an increased branching, and an increasing unsaturation in the fatty acid chain. Harrington, K. J. Biomass, 9:1-17 (1986). Additional cold flow properties and other biofuel characteristics influenced by FAME have been shown previously. See, e.g., Knothe, Fuel Processing Tech., 86:1059-1070 (2005), hereby incorporated by reference in its entirety.

In one aspect, the OMM FAME profile comprises a reduced mass fraction of one or more of the following saturates: C16:0, C18:0, C20:0, C22:0, and/or C24:0. In other embodiments, the OMM disclosed herein comprise FAME profiles comprising a lower mass fraction (m/m) of long chain saturated fatty acids than the FAME profile of the unmodified microorganism when grown in culture. In other embodiments, the OMM comprise a lower mass fraction (m/m) and/or volume fraction (m/m) of long chain saturated fatty acids than the unmodified microorganism when grown in culture. In yet another embodiment, the FAME profile of the OMMs comprise a combined arachidic acid (C20:0), behenic acid (C22:0), and lignoceric acid (C24:0) mass fraction (m/m) of about 2 percent or less when grown in culture.

In certain embodiments, the OMM disclosed showed significant reductions in palmitic (C16:0) and stearic acid (C18:0) FAME when compared to the FAME of the unmodified microorganism. In these embodiments, the FAME profile of the OMM comprises a combined palmitic acid and stearic acid mass fraction (m/m) that is lower than the FAME profile of the unmodified microorganism when grown in culture. In some embodiments, the FAME profile of the modified microorganism comprises a combined palmitic acid and stearic acid mass fraction (m/m) of about 12.5, 10, or 8 percent or less when grown in culture.

In yet another embodiment, the FAME profile of the OMM comprises a combined myristic acid and stearic acid mass fraction (m/m) that is lower than the FAME profile of the unmodified microorganism when grown in culture.

In yet other embodiments, the FAME profile of the OMM comprises a combined arachidic acid, behenic acid, and lignoceric acid mass fraction (m/m) of about 2 percent or less when grown in culture.

In addition and/or the alternative, the FAME profile can include: about 30 percent oleic acid to about 90 percent oleic acid; about 50 percent oleic acid to about 70 percent oleic acid; about 60 percent oleic acid, and/or the like, all on a mass fraction basis. The profile can include about 10 percent linoleic acid to about 70 percent linoleic acid; about 30 percent linoleic acid to about 50 percent linoleic acid; about 15 percent linoleic acid to about 35 percent linoleic acid; about 40 percent linoleic acid, and/or the like.

According to some embodiments, the FAME profile can include: about 1 percent palmitic acid to about 10 percent palmitic acid; about 0.5 percent stearic acid to about 2.5 percent stearic acid; about 50 percent oleic acid to about 70 percent oleic acid; about 15 percent linoleic acid to about 35 percent linoleic acid; and/or about 6 percent linolenic acid to about 12 percent linolenic acid.

According to some embodiments, the FAME profile can include: about 0 percent myristic acid to about 1.5 percent myristic acid; about 1 percent palmitic acid to about 10 percent palmitic acid; about 0.5 percent stearic acid to about 2.5 percent stearic acid; about 0 percent arachidic acid to about 1.5 percent arachidic acid; about 0 percent behenic acid to about 1.5 percent behenic acid; about 0 percent lignoceric acid to about 2 percent lignoceric acid; about 0 percent palmitoleic acid to about 1 percent palmitoleic acid; about 50 percent oleic acid to about 70 percent oleic acid; about 0 percent eicosenoic acid to about 3 percent eicosenoic acid; about 0 percent erucic acid to about 5 percent erucic acid; about 15 percent linoleic acid to about 35 percent linoleic acid; and/or about 6 percent linolenic acid to about 12 percent linolenic acid.

Unsaturated fatty acids, also referred to as “unsaturates”, are distinguished as monounsaturated or polyunsaturated, depending on the number of double bonds. The unsaturated, especially polyunsaturated, fatty esters have lower melting points, which are desirable for improved cold flow properties. However, a higher quantity of polyunsaturated fatty acids, such as 18:2, 18:3, can contribute to the reduced oxidative stability through accelerated autoxidation at the higher number of allylic and bis-allylic positions on the fatty acid backbone. The relative rates of autoxidation of the unsaturates given in the literature clearly demonstrate this point: 1 for oleates (18:1), 41 for linoleates (18:2), and 98 for linolenates (18:3). Frankel, Lipid Oxidation. 2nd Edn. The Oily Press, Bridgewater (UK) 2005. EN14214 sets the maximum mass fraction of polyunsaturated methyl esters with greater than 4 double bonds in the FAME profile as 1.0 percent or lower. This specification serves to eliminate fish oil as biofuel feedstock. With a higher content of methylene-interrupted double bonds, fish oil FA are even more prone to oxidation than linolenic acid and its esters.

The OMM FAME can have any desirable profile and/or characteristics, such as generally suitable for biofuel production. According to some embodiments, the fatty acids can include a suitable amount and/or percent fatty acids with four or more double bonds on a mass basis. In the alternative, the fatty acids can include a suitable amount and/or percent fatty acids with three or more double bonds, with two or more double bonds, with one or more double bonds, and/or the like.

In one embodiment, the OMM comprises a FAME profile comprising a polyunsaturated methyl ester mass fraction (m/m) of 1.0 percent or less. In another embodiment, the FAME profile of the OMM comprises a lower mass fraction (m/m) of polyunsaturated methyl esters than the FAME profile of the unmodified microorganism when grown in culture.

In one aspect, the genetic modification alters the mass fraction (m/m) of individual FA and/or FAME components of the disclosed OMM FAME profile. The genetic modification may affect one or more individual FA components. The genetic modification may affect all or a subset of individual FA components. In some embodiments, the OMM FAME profile comprises FAMEs with advantageous properties, such as esters of decanoic, palmitoleic, and oleic acids. In other embodiments, FAMEs with problematic properties are kept to a minimum.

In a certain embodiment, the individual FA component is palmitic acid (16:0). In a specific embodiment, the OMM FAME profile comprises a palmitic acid mass fraction (m/m) of between 1.0 and 10.0 percent. In other embodiments, the palmitic acid mass fraction comprises about 16, 11, or 10 percent or less when grown in culture.

In certain embodiments, the FAME profile of the OMM comprises a lower mass fraction (m/m) of palmitic acid than the FAME profile of the unmodified microorganism when grown in culture. In one embodiment, the FAME profile of the OMM comprise a palmitic acid mass fraction (m/m) that is at least about 10 percent less than the FAME profile of the unmodified microorganism when grown in culture. In other embodiments, the palmitic acid mass fraction is at least 20, 40, or 60 percent less. In yet another embodiment, the OMM FAME profile comprises a mass fraction (m/m) of palmitic acid that is about 1.0 percent or more when grown in culture.

In another embodiment, the genetic modification affects the stearic acid component of the OMM FAME profile. In specific embodiments, the FAME profile of the OMM comprises a mass fraction (m/m) of stearic acid that is between about 0.5 percent and about 2.5 percent when grown in culture.

In some embodiments, the FAME profile of the OMM comprises a lower mass fraction (m/m) of stearic acid than the FAME profile of the unmodified microorganism when grown in culture. In other embodiments, the OMM FAME profile comprises at least about 10 percent less stearic acid than the unmodified microorganism. In yet other embodiments, the OMM FAME profile comprises at least about 2.5 percent less stearic acid. In further embodiments, the OMM FAME profile comprises at least about 0.5 percent less.

In other embodiments, the FAME profile of the OMM comprises a higher mass fraction (m/m) of stearic acid than the FAME profile of the unmodified microorganism when grown in culture.

In another embodiment, the genetic modification affects the myristic acid component of the OMM FAME profile. In some embodiments, the FAME profile of the OMM comprises a lower mass fraction (m/m) of myristic acid than the FAME profile of the unmodified microorganism when grown in culture. In other embodiments, the OMM FAME profile comprises a myristic acid mass fraction (m/m) of about 1.5 percent or less when grown in culture. In other embodiments, the OMM FAME profile comprises a myristic acid mass fraction (m/m) of about zero when grown in culture.

In another embodiment, the genetic modification affects the arachidic acid component of the OMM FAME profile. In some embodiments, the FAME profile of the OMM comprises a lower mass fraction (m/m) of arachidic acid than the FAME profile of the unmodified microorganism when grown in culture. In other embodiments, the OMM FAME profile comprises an arachidic acid mass fraction (m/m) of about 1.5 percent or less when grown in culture. In other embodiments, the OMM FAME profile comprises an arachidic acid mass fraction (m/m) of about zero when grown in culture.

In another embodiment, the genetic modification affects the behenic acid component of the OMM FAME profile. In some embodiments, the FAME profile of the OMM comprises a lower mass fraction (m/m) of behenic acid than the FAME profile of the unmodified microorganism when grown in culture. In other embodiments, the OMM FAME profile comprises a behenic acid mass fraction (m/m) of about 1.5 percent or less when grown in culture. In other embodiments, the OMM FAME profile comprises an behenic acid mass fraction (m/m) of about zero when grown in culture.

In another embodiment, the genetic modification affects the lignoceric acid component of the OMM FAME profile. In some embodiments, the FAME profile of the OMM comprises a lower mass fraction (m/m) of lignoceric acid than the FAME profile of the unmodified microorganism when grown in culture. In other embodiments, the OMM FAME profile comprises a lignoceric acid mass fraction (m/m) of about 2.0 or 1.0 percent or less when grown in culture. In other embodiments, the OMM FAME profile comprises a lignoceric acid mass fraction (m/m) of about 0.5 percent when grown in culture.

In another embodiment, the genetic modification affects the palmitoleic acid component of the OMM FAME profile. In some embodiments, the FAME profile of the OMM comprises a lower mass fraction (m/m) of palmitoleic acid than the FAME profile of the unmodified microorganism when grown in culture. In other embodiments, the OMM FAME profile comprises a palmitoleic acid mass fraction (m/m) of about 1.0 or 0.5 percent or less when grown in culture. In other embodiments, the OMM FAME profile comprises a palmitoleic acid mass fraction (m/m) of about 0.4 percent when grown in culture.

In another embodiment, the genetic modification affects the oleic acid component of the OMM FAME profile. In some embodiments, the FAME profile of the OMM comprises a lower mass fraction (m/m) of oleic acid than the FAME profile of the unmodified microorganism when grown in culture. In some embodiments, the OMM comprises an oleic acid mass fraction (m/m) that is at least about 3 percent less than the FAME profile of the unmodified microorganism when grown in culture.

In other embodiments, the OMM FAME profile comprises an oleic acid mass fraction (m/m) of about 70 percent or less when grown in culture.

In other embodiments, the FAME profile of the OMM comprises a higher mass fraction (m/m) of oleic acid than the FAME profile of the unmodified microorganism when grown in culture. In some embodiments the modified microorganism comprises a mass fraction (m/m) of oleic acid that is about 50.0 percent or more when grown in culture. In yet another embodiments, the mass fraction (m/m) of oleic acid is between about 50.0 percent and about 70.0 percent when grown in culture.

In another embodiment, the genetic modification affects the eicosenoic acid component of the OMM FAME profile. In some embodiments, the FAME profile of the OMM comprises a lower mass fraction (m/m) of eicosenoic acid than the FAME profile of the unmodified microorganism when grown in culture. In other embodiments, the OMM FAME profile comprises a eicosenoic acid mass fraction (m/m) of about 3.0 or 1.0 percent or less when grown in culture. In other embodiments, the OMM FAME profile comprises a eicosenoic acid

mass fraction (m/m) of about 0.2 percent or less when grown in culture.

In another embodiment, the genetic modification affects the erucic acid component of the OMM FAME profile. In some embodiments, the FAME profile of the OMM comprises a lower mass fraction (m/m) of erucic acid than the FAME profile of the unmodified microorganism when grown in culture. In other embodiments, the OMM FAME profile comprises a erucic acid mass fraction (m/m) of about 5.0 percent or less when grown in culture. In other embodiments, the OMM FAME profile comprises a erucic acid mass fraction (m/m) of about zero percent when grown in culture.

According to certain embodiments, the genetic modification alters the mass fraction (m/m) of linoleic acid in the FAME profile of the OMM.

In one embodiment, the FAME profile of the OMM comprises a lower mass fraction (m/m) of linoleic acid than the FAME profile of the unmodified microorganism when grown in culture. In another embodiment, the FAME profile of the OMM comprises a mass fraction (m/m) of linoleic acid that is about 35.0 percent or less when grown in culture.

In one embodiment, the FAME profile of the OMM comprises a higher mass fraction (m/m) of linoleic acid than the FAME profile of the unmodified microorganism when grown in culture. In another embodiment, the FAME profile of the OMM comprises a mass fraction (m/m) of linoleic acid that is about 15.0 percent or more when grown in culture.

In one embodiment, the FAME profile of the OMM comprises a mass fraction (m/m) of linoleic acid that is between about 15.0 percent and about 35.0 percent when grown in culture.

The content of linolenic acid methyl ester is restricted in EN14214 because of the propensity of linolenic acid methyl ester to oxidize. However, the 12 percent limit is set so as not to exclude rapeseed oil, which has a high oleic acid content, and is a major biodiesel source in Europe.

In one embodiment, the genetic modification alters the mass fraction (m/m) of linolenic acid in the FAME profile of the OMM. In one embodiment, the FAME profile of the OMM comprising a lower mass fraction (m/m) of linolenic acid than the FAME profile of the unmodified microorganism when grown in culture. In another embodiment, the OMM comprise a FAME profile comprising a mass fraction (m/m) of linolenic acid that is about 12.0 percent or less when grown in culture.

In another embodiment, the FAME profile of the OMM comprising a higher mass fraction (m/m) of linolenic acid than the FAME profile of the unmodified microorganism when grown in culture. In a specific embodiment, the FAME profile of the OMM comprises a mass fraction (m/m) of linolenic acid that is about 6.0 percent or more when grown in culture. In another specific embodiment, the FAME profile of the OMM comprises a mass fraction (m/m) of linolenic acid that is between about 6.0 percent and about 12.0 percent when grown in culture.

### 4. COLD FLOW PROPERTIES

One of the major problems associated with the use of biofuel is poor cold flow properties. Pure biodiesel (B100) has poor cold weather operability properties and would need to be stored in heated tanks in colder climates. Heated tanks are more expensive to install and operate. In B20 or higher blends, biodiesel deteriorates the overall cold flow performance of the blend resulting in significant operability challenges. The use of additives and/or base diesel with excellent cold flow properties (No. 1 diesel) may improve biodiesel blends cold weather handling.

Cold flow properties used for assessing biofuels include, for example, parameters such as cold point (CP), pour point (PP), and cold filter plugging point (CFPP). FAME profiles can directly or indirectly impact cold flow properties of biofuel. For example, melting point is a parameter used to assess the suitability of individual FAMEs. Melting points of FAME generally increase with an increasing number of CH₂ moieties and decrease with an increasing unsaturation. FAME profiles with these properties will crystallize at higher temperatures than their cis-unsaturated counterparts and, as a result, may plug engine filters and fuel lines during winter months in temperate climates. Lee, et al., J. Am. Oil Chem. Soc., 72:1155-1160 (1995). It has been shown that biofuels derived from fats or oils with significant amounts of saturated fatty compounds will display higher CP, PP, and/or CFPP. McCormick, R., et al. Env. Sci. & Tech. 35(9): 1742-1747 (2001). Indeed, animal fats, palm, and coconut oils are more highly saturated, and comprise a higher CP. In contrast, rapeseed methyl esters are less saturated and have superior cold flow properties.

In one aspect, the OMMs disclosed herein produce FAME profiles with improved cold flow properties. In certain embodiments, the improved cold flow property is the cloud point, pour point, and/or cold filter plugging point of the FAME profile. According to some embodiments, the OMM FAME profiles comprise properties that influence cold flow properties, such as for example reduced long chain saturates, reduced total saturates, and/or increased polyunsaturates.

Cold flow properties are also specified in international biofuel standards. In another aspect, the OMM disclosed herein comprise FAME profiles with desirable cold flow properties. The OMM disclosed comprise FAME profiles that meet one or more specifications in accepted biofuel standards. In certain embodiments, the cold flow properties include CP, PP, and/or CFPP. In some embodiments, the OMM FAME profile comprises more desirable cold flow properties than the FAME profile of the unmodified microorganism when grown in culture.

In the United States, ASTM D6751: Standard Specification for Biodiesel Fuel Blend Stock (B100) for Middle Distillates is the main biofuel standard specifying cold flow properties. The cold flow properties of biodiesel (B100) meeting the specifications in biofuel standard ASTM:D6751 depend on the number of carbons in the fatty acid chains, the degree of saturation of the fatty acid chains, and the alcohol to which the fatty acid chains are esterified.

The standard EN590: Automotive fuels. Diesel. Requirements and test methods describes the physical properties that all automotive diesel fuel must meet if it is to be sold in the European Union, Croatia, Iceland, Norway and Switzerland. For climate-dependent requirements, options are given to allow for seasonal grades to be set nationally. The standard EN 590 puts diesel fuel into two groups destined for specific climatic environments, such as temperate or arctic climates. EN590 comprises specifications for CP (EN 23015/IP 219) and CFPP (EN 116/IP 309), all of which are hereby incorporated by reference in their entirety.

In certain embodiments, the cold flow property is the cloud point. In one embodiment, the FAME profile of the OMM comprises a cloud point less than about 4 degrees Celsius. In another embodiment, the FAME profile of the OMM comprises a cloud point less than about −3 degrees Celsius. In yet another embodiment, the FAME profile of the OMM comprises a cloud point less than about −18 degrees Celsius. In other embodiments, the FAME profile of the OMM comprises a cloud point of any of the strains listed in Tables 4-7.

Cloud point is of importance in that it defines the temperature at which a cloud or haze of crystals appears in the fuel under prescribed test conditions which generally relate to the temperature at which crystals begin to precipitate from the fuel in use. Further information is contained within ASTM D975, incorporated by reference herein.

ASTM D6751 does not specify a CP limit, and instead a report is required. D6751 notes that it is unrealistic to specify low temperature properties of biodiesel blends that will ensure satisfactory operation at all ambient conditions in all storage situations. This is due in part to the strongly varying weather conditions in the United States. According to ASTM D6751, test method D2500 can be used to determine CP. However, D6751 provides for several other methods, including D5771, D5772, D5773, D7397, D3117, and/or AOCS Standard Procedure Ck 2-09, all of which are incorporated by reference in their entirety. EN23015: Petroleum products—Determination of cloud point can also be used to calculate CP, and is hereby incorporated by reference in its entirety.

One standard method of determining CP is described in ASTM D5771. This test method uses an optical detection stepped cooling method. It covers the range of temperatures from −40° C. to 49° C. with a temperature resolution of 0.1° C. A microprocessor controlled cloud point apparatus continuously controls the temperature of one or more independent test cells and detects the appearance of the cloud point at the bottom of the beaker. The detection of cloud point is done using a light emitter on one side and light receiver at the other side of the beaker. The control of temperature is governed by the cooling circulation bath. To avoid moisture in the sample, the sample is filtered through dry lint-free filter paper, until the fuel is clear.

ASTM D5772 uses the linear cooling rate method for detection of the cloud point of biodiesel and diesel fuel. It consists of an automatic cloud point apparatus that has a microprocessor-controlled measuring unit. The unit is capable of cooling the sample, optically observing the cloud point and recording the temperature with a resolution of 0.1° C. This method uses an optical barrier assembly and test cell that consists of a light transmitter and light receiver, with the temperature measuring device mounted on the top of the assembly. For the circulating bath, a refrigerator equipped circulation pump is used with a temperature at least 20° C. lower than the expected cloud point of the fuel.

ASTM D5773 uses a constant cooling rate to determine cloud point by an automatic instrument using an optical device to detect crystal formation. It consists of a solid-state thermoelectric device that has semiconductor material called a Peltier device to cool the fuel sample at a constant rate of 1.5+/−0.1° C./min. It uses a light source with wavelength of 660±10 nm positioned at an acute angle with the light reflected off the polished bottom of the specimen cup. A microprocessor uses a temperature sensor with resolution of 0.1° C., to control the cooling of the fuel test. During this period, the sample is continuously illuminated by a light source. An array of optical detectors continuously monitors the sample for the first appearance of a cloud of wax crystals. When wax crystals appear in the fuel, there will be a change in the phase boundaries of the reflected beam. This change indicates the cloud point. D5773 is capable of determining cloud point within a temperature range of −60° C., to +49° C. Results are reported with a temperature resolution of 0.1° C. D5773 has been found to be equivalent to test method D2500, but the D5773 test method determines the cloud point in a shorter period of time than manual method D2500. Less operator time is required to run the test using D5773. Additionally, no external chiller bath or refrigeration unit is needed. In certain embodiments, the ASTM D5773 test method is utilized by Phase Technology's (Rapid City, S. Dak.) line of 70X and 70Xi series analyzers.

In other embodiments, the cold flow property is the pour point. In one embodiment, the FAME profile of the OMM comprises a pour point less than about

−9 degrees Celsius. In another embodiment, the FAME profile of the OMM comprises a pour point greater than −18 degrees Celsius. In yet another embodiment, the FAME profile of the OMM comprises a pour point between about −18 and about −9 degrees Celsius. In other embodiments, the FAME profile of the OMM comprises the pour point of any of the strains listed in Tables 4-7.

Pour point can be calculated using ASTM D5949: Standard test method for pour point of petroleum products (automatic pressure pulsing method), which is hereby incorporated by reference. ASTM D5949 uses automatic apparatus and yields pour point results in a format similar to the manual method (ASTM D97) when reporting at a 3° C. The D5949 test method determines the pour point in a shorter period of time than manual method D97. Less operator time is required to run the test using this automatic method. Additionally, no external chiller bath or refrigeration unit is needed. D5949 is capable of determining pour point within a temperature range of −57° C., to +51° C. Results can be reported at 1° C. or 3° C., testing intervals. This test method has better repeatability and reproducibility than manual method D97.

In certain embodiments, the cold flow property is the cold filter plugging point. In one embodiment, the FAME profile of the OMM comprises a CFPP less than about zero degrees Celsius. In another embodiment, the FAME profile of the OMM comprises a cold filter plugging point less than about −5 degrees Celsius. In yet another embodiment, the FAME profile of the OMM comprises a cold filter plugging point less than about −10 degrees Celsius. In other embodiments, the FAME profile of the OMM comprises the CFPP of any of the strains listed in Tables 4-7. In other embodiments, the FAME profile of the OMM comprises a CFPP of about 0, 1, or −17 degrees Celsius.

Cold filter plugging point (CFPP) is now often used instead of CP as the criterion to predict the low temperature performance of biofuels. The CFPP is the lowest temperature at which fuel will still flow through a specific filter. All diesel fuels contain wax. Normally the wax is a liquid in solution in the fuel. It is an important component because it gives the fuel a good cetane value. However, when a fuel gets cold the wax will crystalize, and the crystals can block engine fuel filters. If the temperature is sufficiently low to crystallize a lot of wax the engine will stop through fuel starvation. Because removing the wax during refining reduces cetane the amount of wax in diesel is limited by the season. Fuel specifications are set at levels that ensure most users will be free of wax problems most of the time.

In the United States, CFPP is calculated using ASTM D6371:Standard Test Method for Cold Filter Plugging Point of Diesel and Heating Fuels, which is technically equivalent to test methods IP 309 and the European standard EN116: Diesel and domestic heating fuels—Determination of cold filter plugging point, all of which are hereby incorporated by reference in their entirety.

Other test methods for the cold flow properties of conventional diesel fuel exist, such as for example the low-temperature flow test (LTFT) used in ASTM D4539. These methods have also been used to evaluate biofuels, and can also be used to determine cold flow properties of the OMM disclosed herein.

### 5. OLEAGINOUS MODIFIED MICROORGANISMS (OMM)

As disclosed herein, genetic modifications were introduced into oleaginous microorganisms to generate novel oleaginous modified microorganisms (hereafter “OMM”) comprising novel FAME profiles. In some embodiments, the generated OMM FAME profile was desirable for biofuel production.

In one aspect, mutagenesis of fatty acid-producing cells followed by screening for altered levels of FA production generates novel oleaginous microorganisms that produce desirable FAME profiles. These significant and unexpected improvements may result from for example a higher flux of carbon to fatty acids, or any other mechanism. For some microorganisms, the novel FAME profile may result from a mechanism that is not yet characterized.

Disclosed herein is a oleaginous modified microorganism, or OMM, suitable for production of renewable materials. In certain embodiments, the microorganisms disclosed comprise a genetic modification. In some embodiments, the modification is a genetic modification not present in an unmodified microorganism.

The genetic modification can be introduced by many methods. In certain embodiments, the genetic modification is introduced by genetic engineering. In other embodiments, the genetic modification is introduced by random mutagenesis.

In particular embodiments, the modification affects FA synthesis, which is a well-characterized pathway in many microorganisms, such as yeast. In some embodiments, the modification affects one or more genes encoding a protein that contributes and/or controls FA synthesis. In other embodiments, the modification affects one or more regulatory genes that encode proteins that control FA synthesis. In still other embodiments, the modification affects one or more non-coding regulatory regions. In other embodiments, one or more genes is up-regulated or down-regulated such that FA production is decreased or increased. The modification may affect other biological mechanisms, such as mRNA stability, post-translational modifications, and/or the like. The disclosed list of potential mechanisms and/or genes affected by the genetic modification is merely exemplary and is not intended to be limiting in scope.

Modified genes include, for example, branch points in the metabolic pathway of fatty acids. In other embodiments, the gene is up-regulated or down-regulated such that FA production is increased.

Up-regulation of genes encoding the following enzymes comprise non-limiting examples of genes that may be affected by the modification. One example is pyruvate dehydrogenase, which is involved in converting pyruvate to acetyl-CoA. Up-regulation of pyruvate dehydrogenase can increase production of acetyl-CoA, and thereby increase FA synthesis. Another example is acetyl-CoA carboxylase, which catalyzes the initial step in fatty acid synthesis. Accordingly, this enzyme can be up-regulated to increase production of fatty acids. Another example is acyl carrier protein (ACP), which carries the growing acyl chains during fatty acid synthesis. Another example is glycerol-3-phosphate acyltransferase, which catalyzes the rate-limiting step of fatty acid synthesis. Up-regulation of these exemplary enzymes could increase FA production and/or synthesis.

Down-regulation of genes encoding the following enzymes comprise non-limiting examples of genes that may be affected by the modification. One example is citrate synthase, which consumes acetyl-CoA as part of the tricarboxylic acid (TCA) cycle. Down-regulation of citrate synthase can force more acetyl-CoA into the FA synthetic pathway.

In one embodiment, the OMM comprises a genetic modification, wherein the genetic modification affects pyruvate dehydrogenase, acetyl-CoA carboxylase, acyl carrier protein, glycerol-3 phosphate acyltransferase, citrate synthase, stearoyl-ACP desaturase, glycerolipid desaturase, fatty acyl-ACP thioesterase, a fatty acyl-CoA reductase, a fatty aldehyde reductase, a fatty acyl-CoA/aldehyde reductase, and/or a fatty aldehyde decarbonylase.

In another embodiment, the OMM comprises a genetic modification, wherein the genetic modification alters the gene expression of pyruvate dehydrogenase, acetyl-CoA carboxylase, acyl carrier protein, glycerol-3 phosphate acyltransferase, citrate synthase, stearoyl-ACP desaturase, glycerolipid desaturase, fatty acyl-ACP thioesterase, a fatty acyl-CoA reductase, a fatty aldehyde reductase, a fatty acyl-CoA/aldehyde reductase, and/or a fatty aldehyde decarbonylase.

Any species of organism that produces suitable lipid or hydrocarbon can be used, although microorganisms that naturally produce high levels of suitable lipid or hydrocarbon are preferred. Production of hydrocarbons by microorganisms is reviewed by Metzger et al. Applied Microbio. Biotech. 66: 486-496 (2005), as incorporated by reference.

In a certain embodiment, the disclosed oleaginous modified microorganism is a yeast. Examples of gene mutations in oleaginous yeast can be found in the literature (see Bordes et al, J. Microbio. Methods, 70(3):493-502 (2007)). In certain embodiments, the yeast belongs to the genus Rhodotorula, Pseudozyma, or Sporidiobolus. Examples of oleaginous yeast that can be used include, but are not limited to the following oleaginous yeast: Candida apicola, Candida sp., Cryptococcus curvatus, Cryptococcus terricolus, Debaromyces hansenii, Endomycopsis vernalis, Geotrichum carabidarum, Geotrichum cucujoidarum, Geotrichum histendarum, Geotrichum silvicola, Geotrichum vulgare, Hyphopichia burtonii, Lipomyces lipofer, Lypomyces orentalis, Lipomyces starkeyi, Lipomyces tetrasporous, Pichia mexicana, Rodosporidium sphaerocarpum, Rhodosporidium toruloides. Rhodotorula aurantiaca, Rhodotorula dairenensis, Rhodotorula diffluens, Rhodotorula glutinus, Rhodotorula gracilis, Rhodotorula graminis, Rhodotorula minuta, Rhodotorula mucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri, Trichosporon montevideense, Trichosporon pullulans, Trichosporon sp., Wickerhamomyces canadensis, Yarrowia lipolytica, and Zygoascus meyerae.

In some embodiments, a microorganism producing a lipid or a microorganism from which a lipid can be extracted, recovered, or obtained, is a fungus. Examples of fungi that can be used include, but are not limited to the following genera and species of fungi: Mortierella, Mortierrla vinacea, Mortierella alpine, Pythium debaryanum, Mucor circinelloides, Aspergillus ochraceus, Aspergillus terreus, Pennicillium iilacinum, Hensenulo, Chaetomium, Cladosporium, Malbranchea, Rhizopus, and Pythium.

In other embodiments, the yeast belongs to the genus Sporidiobolus pararoseus. In a specific embodiment, the disclosed microorganism is the microorganism corresponding to ATCC Deposit No. PTA-13344 (Strain MK29404 Dry-1-321C). In another specific embodiment, the disclosed microorganism is the microorganism corresponding to ATCC Deposit No. PTA-13346 (Strain MK29404 248A).

In other embodiments, the yeast belongs to the genus Rhodotorula ingeniosa. In a specific embodiment, the disclosed microorganism is the microorganism corresponding to ATCC Deposit No. PTA-13345 (Strain MK29794 30D). In another specific embodiment, the disclosed microorganism is the microorganism corresponding to ATCC Deposit No. PTA-13347 (Strain MK29794 117D).

In other embodiments, the yeast belongs to the genus Pseudozyma rugulosa or Pseudozyma aphidis. In a specific embodiment, the disclosed microorganism is the microorganism corresponding to ATCC Deposit No. PTA-13342 (Strain MK28428 8-500-3A). In another specific embodiment, the disclosed microorganism is the microorganism corresponding to ATCC Deposit No. PTA-13343 (Strain MK28428 149G).

According to certain embodiments, the oleaginous microorganism is grown in culture, such as for example during manufacture. In some embodiments, such as when the OMM properties need to be compared to the unmodified microorganism, the culture of the OMM comprises substantially similar conditions as the culture of the unmodified microorganism. In certain embodiments, these properties include FAME profile, cold flow properties, and other properties known to evaluate the usability of biofuels. In certain embodiments, the fermentation broth of these cultures comprise a biomass of at least about 50 grams cellular dry weight per liter.

Microorganisms can be cultured both for purposes of conducting genetic manipulations and for subsequent production of hydrocarbons (e.g., lipids, fatty acids, aldehydes, alcohols, and alkanes). The former type of culture is conducted on a small scale and initially, at least, under conditions in which the starting microorganism can grow. For example, if the starting microorganism is a photoautotroph the initial culture is conducted in the presence of light. The culture conditions can be changed if the microorganism is evolved or engineered to grow independently of light. Culture for purposes of hydrocarbon production is usually conducted on a large scale. In certain embodiments, during culture conditions a fixed carbon source is present. The culture can also be exposed to light at various times during culture, including for example none, some, or all of the time.

For organisms able to grow on a fixed carbon source, the fixed carbon source can be, for example, glucose, fructose, sucrose, galactose, xylose, mannose, rhamnose, N-acetylglucosamine, glycerol, floridoside, and/or glucuronic acid. The one or more carbon source(s) can be supplied at a concentration of at least about 50 μM, at least about 100 μM, at least about 500 μM, at least about 5 mM, at least about 50 mM, and at least about 500 mM, of one or more exogenously provided fixed carbon source(s). Some microorganisms can grow by utilizing a fixed carbon source such as glucose or acetate in the absence of light. Such growth is known as heterotrophic growth.

Other culture parameters can also be manipulated. Non-limiting examples include manipulating the pH of the culture media, the identity and concentration of trace elements. and other media constituents. Culture media may be aqueous, such as containing a substantial portion of water.

### 6. BIOFUELS

According to some embodiments, disclosed is a biofuel suitable for use in a compression engine. In certain embodiments, the biofuel is biodiesel.

In one aspect, the biofuel is produced from OMM disclosed herein. In some embodiments, biofuel includes a FAME profile having about 50 percent to about 70 percent oleic acid on a weight percent of total fatty acids basis, and/or about 15 percent to about 35 percent linolenic acid on weight percent of total fatty acids basis, where the biofuel is produced from a microorganism. In other embodiments, the biofuel comprises a desirable FAME profile.

According to one embodiment, the biological oil comprises fatty acids made by any of the methods disclosed herein.

According to one embodiment, the invention includes a biofuel made from any of the biological oils disclosed herein.

According to one embodiment, the invention includes a biofuel suitable for use in a compression engine. The biofuel comprising a fatty acid methyl ester profile of about 50 percent to about 70 percent oleic acid on a weight percent of total fatty acids basis, about 15 percent to about 35 percent linoleic acid on a weight percent of total fatty acids basis, and about less than about 10 percent palmitic acid on a weight percent of total fatty acid basis, where the biofuel is produced from an oleaginous microorganism.

According to one embodiment, the fatty acid methyl ester profile derives from lipids produced by an organism from the kingdom stramenopile, the kingdom fungi, or combinations thereof.

According to some embodiments, the invention is directed to an engine operating on a biofuel made from the any of the biological oils disclosed within this specification.

This disclosure also includes production of microbial lipids and production of biofuel and/or biofuel precursors using the fatty acids contained in those lipids. This disclosure provides for microorganisms that produce lipids suitable for biodiesel production and/or nutritional applications at a very low cost.

According to some embodiments, the disclosure can include a method of producing biological oils. The method can include producing or growing a microorganism as disclosed herein. The microorganism can include and/or have within a lipid containing fatty acids and/or a quantity of lipids containing fatty acids. In the alternative, the organism can excrete and/or discharge the biological oil.

The method can further include any suitable additional actions, such as extracting and/or removing the lipid containing fatty acids by cell lysing, applying pressure, solvent extraction, distillation, centrifugation, other mechanical processing, other thermal processing, other chemical processing, and/or the like. In the alternative, the producing microorganism can excrete and/or discharge the lipid containing fatty acids from the microorganism without additional processing.

In another aspect, disclosed are methods of producing a biofuel precursor. In certain embodiments, the methods comprise culturing the microorganisms as described and collecting the fermentation broth produced by the microorganism. The biofuel precursor can be produced using any of the microorganisms described herein. In some embodiments, the biofuel precursor is a biological oil. The biofuel precursor can be extracted as described herein or by any other suitable technique. If necessary, further chemical processing of extracted lipids and/or biological oils into biofuel precursors can be performed. In some embodiments, the method further comprises extracting fatty acids from the microorganism and reacting the fatty acids to produce a biofuel.

Also disclosed are methods for producing a biofuel. In certain embodiments, the method comprises supplying a carbon source and converting the carbon source to fatty acids within the microorganisms as described. Certain described microorganisms should be cultured to a specific cell density prior to extraction of lipids, oils, biofuels, or biofuel precursors. In certain embodiments, the disclosed method comprises culturing the microorganism to a cell density of at least about 50 grams cellular dry weight per liter in a fermentation broth. In one embodiment, the biofuels or biofuel precursors of the method is produced with any of the modified microorganisms as disclosed herein. In one embodiment, the microorganism is a yeast. In other embodiments, the disclosed method comprises culturing the microorganism to a cell density of at least about 10, 20, 30, 40, 60, 70, 80, 90, 100, 200, 300, 400, 500, 1000 or more grams per liter in a fermentation broth.

A biofuel produced by the described methods is also disclosed. The biofuel may be derived from any of the biofuel precursors or biological oils or lipids as produced by the disclosed methods or microorganisms. The biofuel precursor or biological oil can be further processed into the biofuel with any suitable method, such as esterification, transesterification, hydrogenation, cracking, and/or the like. In the alternative, the biological oil can be suitable for direct use as a biofuel. Esterification refers to making and/or forming an ester, such as by reacting an acid with an alcohol to form an ester. Transesterification refers to changing one ester into one or more different esters, such as by reaction of an alcohol with a triglyceride to form fatty acid esters and glycerol, for example. Hydrogenation and/or hydrotreating refer to reactions to add hydrogen to molecules, such as to saturate and/or reduce materials.

In another aspect, disclosed are methods of powering a vehicle by combusting a biofuel in an internal combustion engine. The biofuel can be produced by any of the described methods or by any of the disclosed microorganisms.

In another aspect, disclosed is a biofuel suitable for use in compression engines. The biofuel can be produced by any of the described methods or by any of the disclosed microorganisms.

Increasing interest is directed to the use of hydrocarbon components of biological origin in fuels, such as biodiesel, renewable diesel, and jet fuel, since renewable biological starting materials that may replace starting materials derived from fossil fuels are available, and the use thereof is desirable. There is an urgent need for methods for producing hydrocarbon components from biological materials. The present disclosure fulfills this need by providing methods and microorganisms suited for production of biodiesel, renewable diesel, and jet fuel using the lipids generated by the methods described herein as a biological material to produce biodiesel, renewable diesel, and jet fuel.

After extraction, lipid and/or hydrocarbon components recovered from the microbial biomass described herein can be subjected to chemical treatment to manufacture a fuel for use in diesel vehicles and jet engines. One example is that biodiesel can be produced by transesterification of triglycerides contained in oil-rich biomass. Lipid compositions can be subjected to transesterification to produce long-chain fatty acid esters useful as biodiesel. Thus, in another aspect of the present disclosure a method for producing biodiesel is provided. In a certain embodiment, the method for producing biodiesel comprises the steps of (a) cultivating a lipid-containing microorganism using methods disclosed herein (b) lysing a lipid-containing microorganism to produce a lysate, (c) isolating lipid from the lysed microorganism, and (d) transesterifying the lipid composition, whereby biodiesel is produced. Transesterification can include use of any suitable alcohol, such as methanol, ethanol, propanol, butanol, and/or the like.

Methods for growth of a microorganism, lysing a microorganism to produce a lysate, treating the lysate in a medium comprising an organic solvent to form a heterogeneous mixture and separating the treated lysate into a lipid composition have been described above and can also be used in the method of producing biodiesel.

### 7. RENEWABLE MATERIAL PRODUCTION

The production of renewable materials, including biological oils, from sources such as plants (including oilseeds), microorganisms, and animals needed for various purposes. For example, it is desirable to increase the dietary intake of many beneficial nutrients found in biological oils. Particularly beneficial nutrients include fatty acids such as omega-3 and omega-6 fatty acids and esters thereof. Because humans and many other animals cannot directly synthesize omega-3 and omega-6 essential fatty acids, they must be obtained in the diet. Traditional dietary sources of essential fatty acids include vegetable oils, marine animal oils, fish oils and oilseeds. In addition, oils produced by certain microorganisms have been found to be rich in essential fatty acids. In order to reduce the costs associated with the production of beneficial fatty acids for dietary, pharmaceutical, and cosmetic uses, there exists a need for a low-cost and efficient method of producing biological oils containing these fatty acids.

In certain embodiments, the oleaginous microorganism produces a renewable material. The renewable materials as disclosed herein can be used for the manufacture of a food, supplement, cosmetic, or pharmaceutical composition for a non-human animal or human. Renewable materials can be manufactured into the following non-limiting examples: food products, pharmaceutical compositions, cosmetics, and industrial compositions. In certain embodiments, the renewable material is a biofuel or biofuel precursor.

A food product is any food for animal or human consumption, and includes both solid and liquid compositions. A food product can be an additive to animal or human foods, and includes medical foods. Foods include, but are not limited to, common foods; liquid products, including milks, beverages, therapeutic drinks, and nutritional drinks; functional foods; supplements; nutraceuticals; infant formulas, including formulas for pre-mature infants; foods for pregnant or nursing women; foods for adults; geriatric foods; and animal foods. In some embodiments, the microorganism, renewable material, or other biological product disclosed herein can be used directly as or included as an additive within one or more of: an oil, shortening, spread, other fatty ingredient, beverage, sauce, dairy-based or soy-based food (such as milk, yogurt, cheese and ice-cream), a baked good, a nutritional product, e.g., as a nutritional supplement (in capsule or tablet form), a vitamin supplement, a diet supplement, a powdered drink, a finished or semi-finished powdered food product, and combinations thereof.

In certain embodiments, the renewable material is a biological oil. In certain embodiments, the renewable material is a saturated fatty acid. In other embodiments, the renewable material is a FAME.

The modified oleaginous microorganisms described herein can be highly productive in generating renewable materials as compared to unmodified counterpart microorganisms. Microorganism renewable material productivity is disclosed in pending U.S. patent application Ser. No. 13/046,065 (Pub. No. 20120034190, filed Mar. 11, 2011), which is herein incorporated by reference in its entirety. In other embodiments, the application discloses methods of producing renewable materials. Methods of producing renewable materials is disclosed in pending U.S. patent application Ser. No. 13/046,065 (Pub. No. 20120034190, filed Mar. 11, 2011), which is herein incorporated by reference in its entirety. Each reference cited in this disclosure is hereby incorporated by reference as if set forth in its entirety.

### EXAMPLES

The following examples are offered to illustrate, but not to limit, the claimed invention.

### Example 1

### Strain Mutagenesis

The strains selected for mutagenesis work were MK29404, a strain of the yeast species Sporidiobolus pararoseus, and MK29794, a strain of the yeast species Rhodotorula ingeniosa, and MK28428, a strain of the yeast species Pseudozyma rugulosa. All of these strains have fatty acid profiles at are too high in saturated fatty acids and need to be closer to a rapeseed-like profile.

Genetic modifications were introduced into these strains by standard UV light, X-Ray irradiation and chemical mutagenesis. To determine the appropriate level of exposure to the different mutagens, kill curves were conducted on each strain and each mutagen. UV light, X-ray irradiation and a chemical mutagen (nitrosoguanidine) were used for each strain.

Briefly, cells were plated onto agar media plates and exposed to a range a UV irradiation dose of 350-475 μjoules. X-ray mutagenesis was conducted by plating cells onto agar media plates and exposing them to X-ray irradiation for 30 min or 1 hour. Chemical mutagenesis was conducted by mixing cells of the unmodified strain with varying levels of nitrosoguanidine for 1 hour. Levels of 20 and 40 μg/ml were used for subsequent generation of mutants.

Mutagenized cells were grown on agar plates with standard Biofuels Growth Media (BFGM) (as detailed in U.S. patent application Ser. No. 13/046,065 (Pub. No. 20120034190). The BFGM media was used at ¼× the full-strength concentration, except for the nitrogen and phosphate, (MSG monohydrate, (NH4)2SO4, Tastone 154, KH2PO4) which were used at 1/16 the concentration of the full strength media. The components of the media are depicted in Table 3. This concentration of media, called 1/16 BFGM, allowed significant fat accumulation but prevented the colonies from overgrowing and merging together.

Numerous colonies were harvested for FAME profile analysis. The colony FAME procedure is semi-quantitative with only the fatty acid profile of the sample determined. To harvest colonies, the cells from individual colonies are removed from the agar surface using a sterile loop and the biomass is transferred to a screw cap glass tube containing 2 mls of a 0.1M solution of sodium methoxide in methanol and heated at 11° C. for 5 minutes. One ml of hexane is added, the tube is vortexed and the hexane layer is separated for GC analysis. The percentage of each fatty acid in the sample is calculated from the peak areas for each fatty acid. This procedure has been shown to consistently give FAME profiles similar to the standard FAME procedure. The mutagenesis successfully altered the FAME profile of the microorganisms as compared to unmodified microorganisms.

TABLE 3
Components of 1/16 BFGM Media.

| | Amount per liter | |
|---|---|---|
| Component | | |
| NaCl | 0.625 | g |
| KCl | 1 | g |
| MgSO4•7H₂O | 5 | g |
| (NH4)2SO4 | 0.0125 | g |
| CaCl2 2H2O | 0.29 | g |
| MSG monohydrate | 0.125 | g |
| Tastone 154 | 0.125 | g |
| HEPES (100 mM) pH 7 | 23.8 | g |
| KH2PO4 | 0.00625 | g |
| Sucrose | 50 | g |
| Agar | 15 | g |
| Trace Metal Solution | | |
| Citric Acid | 1.0 | g |
| FeSO4•7H2O | 10.3 | mg |
| MnCl2•4H2O | 3.1 | mg |
| ZnSO4•7H2O | 1.93 | mg |
| CoCl2•6H2O | 0.04 | mg |
| Na2MoO4•2H2O | 0.04 | mg |
| CuSO4•5H2O | 2.07 | mg |
| NiSO4•6H2O | 2.07 | mg |
| pH to 2.5 with HCl | | |
| Vitamin Solution | | |
| Vitamin B12 | 0.16 | mg |
| Thiamine | 9.75 | mg |
| CaPantothenate | 3.33 | mg |

### Example 2

### FAME Profile Screening

The FAME profile of approximately 1,000 mutant strains of MK29404, MK29494, and MK28428 strains were initially tested using a modified FAME screening procedure. Mutant colonies that showed potentially desirable FAME profiles, such as those similar to rapeseed oil, and/or meeting any of the criteria of Table 2, and/or other FAME profiles of potential interest were selected for further analysis. The colonies showing desirable FAME profiles or otherwise of interest were isolated as follows: Each strain was picked from an agar plate, and inoculated into a shake flask. The shake flask was then used to inoculate another flask (250 ml Erlenmeyer flask containing 50 ml of 1/16 BFGM medium) that was then grown for 5 days at 27 degrees and shaken on a rotary shaker at 200 rpm. After 5 days the flask was harvested by centrifugation, the pellet was washed with water and centrifuged again. The final pellet was freeze dried and fatty acid profile was determined by FAME procedure. An exemplary selection of FAME profiles from several MK29404 mutant strains are shown in Table 4.

TABLE 4
Examples of MK29404 mutant strain FAME profiles
determined by preliminary screening.

| Strain | Mutagen | 16:0 | 18:0 | 18:1 | 18:2 | 18:3 |
|---|---|---|---|---|---|---|
| Control | none | 19.1 | 6.4 | 63.8 | 7.3 | 1.8 |
| 4-49G | UV | 10.8 | 6.9 | 62.8 | 14.2 | 3.5 |
| 16-500-3L | UV | 14.3 | 5.3 | 69.5 | 8.1 | 2.8 |
| C23 | Chemical | 10.7 | 7.7 | 69.9 | 11.7 | 0 |
| 4D5 | X-Ray | 13.4 | 8.6 | 63.0 | 8.3 | 2.5 |
| 4B3 | X-Ray | 12.1 | 5.3 | 50.8 | 22.1 | 9.6 |
| 11E | Chemical | 34.5 | 7.2 | 47.0 | 8.5 | 2.9 |
| 6F | Chemical | 30.4 | 4.6 | 53.3 | 11.7 | 0 |

### Example 3

### FAME Profile Characterization

Select strains were used for further follow up tests in triplicate flasks. Strains were inoculated into a shake flask. The shake flask was then used to inoculate another flask (250 ml Erlenmeyer flask containing 50 ml of 1/16 BFGM medium) that was then grown for 5 days at 27 degrees and shaken on a rotary shaker at 200 rpm. After 5 days the flask was harvested by centrifugation, the pellet was washed with water and centrifuged again. The final pellet was freeze dried and fatty acid profile was determined by known FAME procedures, including the gas chromatography (GC) analysis method described in BN EN14103:2011, hereby incorporated by reference.

Briefly, the media is removed and the lipid inside the cells are converted to esters using an analytical acid-catalyzed esterification protocol. Once the internal lipids are esterified to FAME, they are analyzed by GC with an internal reference standard in order to quantify the amount of lipids recovered. The microorganism FAMEs were run along with a standardized nonadecanoic FAME, from either Fluka (Ref: 74208), Nu Chek (Ref: N-19-M) or Dr. Ehrenstorfer GmbH (ref 15 622 360).

For the GC analysis, the FAME standard and test samples were processed according to EN14103:2011. Briefly, the samples and standard were left in a closed container at ambient temperature or at least 3 h prior to being weighed, in order to limit the water absorption during weighting. Approximately 100 mg of homogenized sample and standard FAME was weighed in a 10 ml tube and diluted with 10 ml toluene. One μl of this solution was analyzed by GC in a Carbowax 20M capillary column, according to the following example conditions:

Column temperature: 60° C. hold for 2 min, programmed at 10° C. min-1 up to 200° C., programmed at 5° C. min-1 up to 240° C., final temperature hold for 7 min Injector temperature & detector temperature: 250° C. Carrier gas flow rate 1-2 ml min-1; injected volume: 1 μl; hydrogen pressure=70 KPa; split flow=100 ml·min-1.

For each sample, two test portions are prepared, each for two chromatographic analyses. For identification, the GC conditions (injected quantity, oven temperature, carrier gas pressure and split flow rate) were adjusted so as to correctly visualize the methyl ester peaks of the lignoceric (C24:0) and nervonic (C24:1) acids. The integration was carried out as from the hexanoic acid methyl ester (C6:0) peak up to that of the nervonic acid methyl ester (C24:1) taking all the peaks identified as FAME into consideration. Therefore the FAME profile is generated. Data from the GC peaks are expressed as mass fraction (m/m) percent of total fatty acids.

FAME profile data for several mutant strains of MK29404, MK29794, and MK28428 are depicted in Table 5.

TABLE 5
Representative Examples of MK29404, 29794, and 28428 Mutant Strain FAME Profiles
Selected for Further Analysis

| Strain | Parent | 16:0 | 18:0 | 18:1 | 18:2 | 18:3 | 20:0 | 20:1 | 22:0 | 24:0 | 16:0 + 18:0 | Total Saturates |
|---|---|---|---|---|---|---|---|---|---|---|---|---|
| Dry 1*^{#} | 29404 | 14.1 | 3.4 | 64.2 | 11.9 | 2.9 | 0.89 | 0.29 | 0.76 | 0.41 | 17.5 | 19.6 |
| 639G*^{#} | 29404 | 9.3 | 2.2 | 60.6 | 20.9 | 2.1 | 0.3 | 0.48 | 0.51 | 0.85 | 11.5 | 13.2 |
| 716J*^{#} | 29404 | 9.3 | 2.3 | 59.3 | 21.7 | 2.3 | 0.22 | 0.46 | 0.45 | 1.12 | 11.6 | 13.4 |
| 321C*^{#} | 29404 | 9.2 | 1.2 | 67.8 | 15.6 | 2.5 | 0.19 | 0.83 | 0.27 | 0.78 | 10.4 | 11.6 |
| 248A*^{#} | 29404 | 6.1 | 0.9 | 67 | 16.4 | 3.1 | 1.69 | 0.63 | 0.62 | 0.29 | 7 | 9.6 |
| 173N*^{#} | 29404 | 8.1 | 2.4 | 70.4 | 11.8 | 2.2 | 0.58 | 0.76 | 0.86 | 1.23 | 10.5 | 13.2 |
| 453H*^{#} | 29404 | 15.1 | 3.8 | 53.6 | 15.9 | 6.3 | 0.82 | 0.32 | 1.12 | 1.29 | 18.9 | 22.1 |
| 161H*^{#} | 29404 | 10.7 | 1.6 | 43.9 | 30.8 | 9.2 | 0.32 | 0.15 | 0.48 | 0.56 | 12.3 | 13.7 |
| 147D* | 29404 | 10.6 | 1.4 | 83.8 | 0 | 0 | 0.32 | 0.82 | 0.5 | 0.58 | 12 | 13.4 |
| 13J* | 29404 | 8.43 | 1.6 | 84.6 | 0 | 0 | 0.34 | 1.1 | 0.51 | 0.72 | 10 | 11.6 |
| 17J* | 29404 | 12.6 | 3.4 | 61.8 | 12.8 | 4.4 | 1.53 | 0.24 | 0.41 | 0.04 | 16 | 18 |
| 72D* | 29404 | 10.3 | 2 | 61.3 | 19.3 | 3.8 | 0.66 | 0.17 | 0.71 | 0 | 12.3 | 13.7 |
| Parent | 29794 | 19 | 4 | 62.4 | 9.1 | 2.3 | 1.12 | 0 | 0.54 | 0 | 23 | 24.7 |
| (wild-type) | | | | | | | | | | | | |
| 117D* | 29794 | 10.3 | 5.8 | 60.2 | 11.3 | 4.1 | 4.14 | 0.66 | 1.15 | 0.28 | 16.1 | 21.7 |
| 30D* | 29794 | 10.8 | 4.6 | 61.1 | 10.6 | 3.9 | 2.58 | 0.32 | 0.98 | 0.34 | 15.4 | 19.3 |
| Parent* | 28428 | 15 | 3.6 | 59.1 | 11 | 0 | 1.7 | 0.06 | 1.34 | 0.73 | 18.6 | 22.4 |
| (wild-type) | | | | | | | | | | | | |
| 8-500-3A* | 28428 | 7.8 | 4.3 | 59.6 | 13.5 | 0 | 1 | 0.3 | 1.25 | 1.22 | 12.1 | 15.6 |
| 149G* | 28428 | 5.5 | 4.5 | 46.1 | 11.8 | 0.1 | 1.07 | 0.31 | 1..23 | 1.32 | 10 | 13.6 |
| 171C* | 28428 | 14.6 | 3.5 | 52.2 | 17.5 | 0.1 | 2 | 0.13 | 1.99 | 1.02 | 18.1 | 23.1 |
| 477* | 28428 | 11.7 | 2.5 | 68.1 | 8.5 | 0 | 1.32 | 0.06 | 0.9 | 0.36 | 14.2 | 16.8 |

*Avg of 3 replicates
^{#}Grown at atmospheric 10% CO2

### Example 4

### Fuel Properties

Selected strains were fermented in large batches for further examined of fuel properties.

Fermentations were conducted in a manner similar to Example 15 from U.S. patent application Ser. No. 13/046,065 (Pub. No. 20120034190), which is incorporated by reference. Strains were cultivated in a 14 liter New Brunswick Scientific BioFlo 3000 fermentor with a carbon (sucrose syrup) and nitrogen (ammonium hydroxide) fed-batch process.

The fermentation was inoculated with 0.5 liters of inoculum culture from a 2 L fernbach flask grown for 2 days at 27 degrees at 200 rpm on a rotary shaker. The media consisted of 10.0 g/L Tastone 154 (yeast extract), 4.5 g/L NaCl, 0.3 g/L CaCl₂*2H₂O, 1.25 g/L MgSO₄*7H2O, 75 g/L Glucose.

The fermentation media included 4 batched media groups. Group A included 6.25 grams NaCl, 4.2 grams (NH4)2SO4, 10 grams yeast extract (T154), 12.66 grams Na2HPO4, and 1.0 milliliters Dow 1520US (antifoam). Group A was autoclaved at 121 degrees Celsius in the fermentor at a volume of approximately 5.0 liters. Group B included 103 milligrams FeSO4*7H2O, 370 milligrams citric acid, 31 milligrams MnCl2*4H2O, 31 milligrams ZnSO4*7H2O, 0.4 milligrams CoCl2*6H2O, 0.4 milligrams Na2MoO4*2H2O, 20.7 milligrams CuSO4*5H₂O, and 20.7 milligrams NiSO4*6H₂O in a volume of approximately 45 milliliters of distilled water. The group B stock solution was autoclaved at 121 degrees Celsius. Group C included 97.5 milligrams thiamine-HCl, 1.6 milligrams vitamin B12, 33.3 milligrams pantothenic acid hemi-calcium salt, and 35.8 micrograms biotin dissolved in approximately 10 milliliters and filter sterilized. Group D included approximately 700 milliliters of sugar syrup obtained from Raceland Raw Sugar Corporation in Louisiana, U.S.A. After the fermentor was cooled to 27 degrees Celsius, groups B, C, and D were added to the fermentor. Using sodium hydroxide and sulfuric acid, the fermentor was pH adjusted to 7.0 and the dissolved oxygen was spanned to 100 percent prior to inoculation. The fermentor volume prior to inoculation was approximately 6.15 liters.

The fermentation was pH controlled utilizing a 0.26 liter solution of 6N ammonium hydroxide at a pH of 7.0. The dissolved oxygen was controlled to maintain at least 20 percent throughout the fermentation using agitation from 357 revolutions per minute to 1200 revolutions per minute, airflow was 8 liters per minute, and oxygen supplementation from 0.0 liters per minute to 5.0 liters per minute. Throughout the fermentation, 5.65 liters of (Raceland) sugar syrup was fed to maintain a total sugar (glucose+fructose+sucrose) concentration less than 80 grams per liter.

After 5 days the biomass was harvested by centrifugation, washed with water, and freeze-dried. Freeze-dried solids were ground to a fine particle size using a standard household coffee grinder. The ground solids (100 grams) were then weighed in a glass beaker, and hexane was added to a 7-7.5% wt/vol solution (1400 ml). The solids were wetted using a high shear mixer.

An overhead air-driven stirrer was used to keep the solids in suspension while the slurry was pumped into the feed reservoir of a Microfluidics homogenizer (Model M110Y). The slurry in the feed reservoir was manually agitated with a metal stir rod to prevent the biomass from settling. The Microfluidizer was set-up for cell disruption with a 200 micron auxiliary processing module and a 100 micron Z-type interaction chamber (G10Z) in series. The material was subjected to one pass through the homogenizer at an operating pressure of 15,000 psi.

The homogenization resulted in cell disruption, and liberation of crude oil, which was solubilized into the hexane phase. The homogenized material was decanted to centrifuge bottles (˜500 ml each) and centrifuged (4800 rpm, 5 minutes) to remove cell debris and media solids from the hexane/crude oil layer. (The solids, representing the hexane extracted biomass is called biomeal.) The lighter hexane/crude oil slurry was decanted off and introduced into a round bottomed flask with a known weight. The flask was attached to a Rotary Evaporator (Buchi) with a water bath temperature of ˜60-70° C. The hexane was evaporated off to a volume of 300-500 mls. The concentrated hexane/oil mixture was then centrifuged (4000 rpm, 10 minutes) to remove fine solids. The hexane/oil supernatant was then evaporated off to a constant weight with the Buchi Rotary Evaporator. The crude oil in the flask was centrifuged a final time (4000 rpm, 10 minutes) to remove any residual particulate matter. The finished crude oil was decanted into a clean storage container, purged with nitrogen and stored refrigerated.

Crude oil was esterified using a sodium methoxide catalyst and methanol at reflux for a minimum of 3 hours. The crude reaction mixture was then neutralized and washed several times with water to remove the residual catalyst, glycerol co-product and unreacted methanol. The isolated methyl ester was subsequently dried overnight in vacuo prior to any cloud and pour point assessment.

A Phase Technologies (Model PSA-70) automated cloud/pour/freeze point apparatus was used for cloud and pour point determination. The cooling capacity of the instrument was facilitated via the use of a re-circulating chiller containing an aqueous ethylene glycol solution set to −5° C. Nitrogen gas was plumbed in at ˜20 psi to aid in determining the pour point. Approximately 150 μL of the respective FAME was pipetted into the instrument's sample cup, ensuring no air bubbles in the cup after dispensing the sample. A sample pre-heat option on the instrument was selected, which initially set the sample cup temperature to 70° C. prior to any cloud and pour point determination. Cloud and pour points were measured in increments of ±0.1° C. and ±3° C. The method is consistent with the international standards for measuring cloud and pour point: ASTM D-5773/ASTM D-5949.

To determine cold filter plugging point temperature (CFPP), a new batch of FAME were prepared from fermentation of selected strains. CFPP was determined using an automated water bath apparatus, and was performed per EN116, an optional test outlined in EN14214, the European Standard Compendia for determining fatty acid methyl ester quality. Briefly, the specimen of the sample is cooled under specified conditions and, at intervals of 1° C., is drawn into a pipet under a controlled vacuum through a standardized wire mesh filter. The procedure is repeated, as the specimen continues to cool, for each 1° C. below the first test temperature. Testing is continued until the amount of wax crystals that have separated out of solution is sufficient to stop or slow down the flow so that the time taken to fill the pipet exceeds 60 s or the fuel fails to return completely to the test jar before the fuel has cooled by a further 1° C. The indicated temperature at which the last filtration was commenced is recorded as the CFPP. Each sample was run in triplicate and an average value is reported CP, PP, and CFPP are rounded to the nearest whole degree (° C.).

The FAME profiles and cold flow fuel properties of the mutant strains after fermentation are shown in Tables 6, 7, and 8.

TABLE 6
FAME Profile and Fuel Properties of Fermented Mutant strains of MK29404.

| | MK29404 Mutant ID | | | | | | | | | | |
|---|---|---|---|---|---|---|---|---|---|---|---|
| | WILD | | Dry- | Dry-1- | Dry-1- | Dry-1- | | | | | |
| FAME Profile: | TYPE | 248A | 173N | 174-D | 321-C | 182-J | Dry-55 | Dry-41 | Dry-1 | CB19 | RME |
| % 14:0 | 0.610 | 0.2 | 0.41 | 0.42 | 0.22 | 0.40 | 0.00 | 0.00 | 0.77 | 1.15 | 0.05 |
| % 16:0 | 15.715 | 6.18 | 8.79 | 11.67 | 9.19 | 9.92 | 14.09 | 15.42 | 16.54 | 21.45 | 4.64 |
| % 16:1 | 0.325 | .67 | 0.46 | 0.73 | 0.35 | 0.31 | 0.51 | 0.40 | 0.45 | 0.90 | 0.22 |
| % 17:0 | 2.676 | 0 | 0.15 | 0.18 | 0.09 | 0.19 | 0.10 | 0.19 | 0.15 | 0.11 | 0.08 |
| % 18:0 | 4.276 | 0.92 | 1.37 | 1.81 | 1.96 | 3.31 | 2.43 | 4.59 | 4.37 | 3.27 | 1.72 |
| % 18:1 n-9 | 63.076 | 68.02 | 74.52 | 79.78 | 75.15 | 74.64 | 69.05 | 67.91 | 0.00 | 0.11 | 3.30 |
| % 18:1 n-7 | 0.000 | 0.00 | 0.00 | 0.00 | 0.20 | 0.00 | 0.09 | 0.07 | 69.22 | 62.75 | 57.31 |
| % 18:2 | 11.272 | 16.71 | 6.73 | 0.05 | 7.20 | 5.84 | 6.09 | 6.33 | 5.24 | 6.74 | 19.80 |
| % 18:3 n-3 | — | 3.19 | 1.23 | 0.07 | 1.14 | 0.52 | 0.00 | 0.46 | 0.54 | 0.72 | 9.55 |
| % 20:0 | 0.861 | 1.14 | 0.60 | 0.53 | 0.35 | 0.50 | 0.93 | 0.79 | 0.85 | 0.49 | 0.60 |
| % 20:1 n-9 | 0.129 | 1.22 | 0.87 | 1.03 | 1.36 | 0.54 | 0.59 | 0.42 | 0.29 | 0.39 | 1.28 |
| % 22:0 | 0.821 | 0.6 | 0.80 | 0.59 | 0.40 | 0.55 | 0.45 | 0.72 | 0.69 | 0.59 | 0.34 |
| % 24:0 | 0.149 | 0.3 | 1.02 | 0.47 | 0.69 | 0.72 | 0.68 | 0.55 | 0.53 | 0.62 | 0.12 |
| % 16:0 + 18:0 | 19.991 | 7.1 | 10.16 | 14.48 | 11.15 | 13.23 | 16.99 | 20.01 | 20.91 | 24.72 | 6.36 |
| % Total Saturates | 22.43 | 8.45 | 13.14 | 15.67 | 12.90 | 15.58 | 19.59 | 22.26 | 23.91 | 27.68 | 7.56 |
| Cloud Point (° C.) | | | 9.2 | 2.9 | 4.6 | 5.8 | 4.8 | 7.0 | 5 | 11 | −3.2 |
| Pour Point (° C.) | | | −6 | −3 | −3 | −3 | 0 | 3 | 3 | 3 | −12 |
| CFPP (° C.) | | | | | | | | | 0 | 1 | −17 |

TABLE 7
FAME Profile and Fuel Properties of Fermented Mutant strains of MK29794.

| | MK29794 Mutant ID | | | | | | | |
|---|---|---|---|---|---|---|---|---|
| | WILD | | | | | | | |
| FAME Profile: | TYPE | K200 dry1 | KDry7 | 33dry1 | Kdry16-1 | Dry-1 | CB19 | RME |
| % 14:0 | 0.93 | 0.78 | 0.72 | 0.72 | 0.77 | 0.00 | 1.15 | 0.05 |
| % 16:0 | 18.97 | 18.11 | 17.17 | 16.17 | 17.35 | 15.24 | 21.45 | 4.64 |
| % 16:1 | 1.03 | 0.90 | 0.87 | 0.87 | 1.00 | 0.33 | 0.90 | 0.22 |
| % 17:0 | 0.08 | 0.11 | 0.10 | 0.09 | 0.10 | 0.11 | 0.11 | 0.08 |
| % 18:0 | 5.02 | 3.61 | 3.72 | 3.93 | 5.34 | 4.54 | 3.27 | 1.72 |
| % 18:1 n-9 | 65.90 | 65.64 | 66.84 | 67.00 | 68.16 | 64.68 | 62.75 | 57.31 |
| % 18:1 n-7 | 0.08 | 0.16 | 0.19 | 0.17 | 0.12 | 0.06 | 0.11 | 3.30 |
| % 18:2 | 4.29 | 5.41 | 5.57 | 5.53 | 2.88 | 5.79 | 6.74 | 19.80 |
| % 18:3 n-3 | 0.27 | 0.66 | 0.80 | 0.73 | 0.18 | 0.89 | 0.72 | 9.55 |
| % 20:0 | 1.12 | 0.83 | 1.03 | 1.29 | 0.85 | 0.84 | 0.49 | 0.60 |
| % 20:1 n-9 | 0.26 | 0.30 | 0.23 | 0.20 | 0.20 | 0.37 | 0.39 | 1.28 |
| % 22:0 | 0.61 | 0.60 | 0.57 | 0.64 | 0.34 | 0.84 | 0.59 | 0.34 |
| % 24:0 | 0.25 | 0.43 | 0.29 | 0.28 | 0.10 | 0.70 | 0.62 | 0.12 |
| % 16:0 + 18:0 | | | | 20.1 | 22.69 | 19.78 | 24.72 | 6.22 |
| % Total Saturates | 26.98 | 24.48 | 23.6 | 23.12 | 23.6 | 22.27 | 27.68 | 7.56 |
| Cloud Point (° C.) | 7.1 | 8.1 | 4.4 | 5.7 | 7.6 | 8.7 | 11 | −3.2 |
| Pour Point (° C.) | 6 | 3 | 6 | 6 | 6 | 3 | 3 | −12 |
| CFPP (° C.) | | | | | | 0 | 1 | −17 |

TABLE 8
FAME Profile and Fuel Properties of Fermented Mutant strains of MK28428.

| | MK28428 Mutant ID | | | | | | | | |
|---|---|---|---|---|---|---|---|---|---|
| | WILD | | | | | | | | |
| FAME Profile: | TYPE | 8-500-3A-1 | 149G | 477H | 155A | 3ZA-LF | Dry-1 | CB19 | RME |
| % 14:0 | 0.698 | 0.740 | 1.447 | 0.58 | 0.89 | 0.84 | 0.77 | 1.15 | 0.05 |
| % 16:0 | 15.265 | 8.029 | 5.888 | 15.67 | 16.16 | 20.23 | 16.54 | 21.45 | 4.64 |
| % 16:1 | 2.828 | 2.326 | 0.689 | 3.00 | 2.32 | 2.35 | 0.45 | 0.90 | 0.22 |
| % 17:0 | 0.000 | 0.000 | 0.000 | 0.11 | 0.10 | 0.11 | 0.15 | 0.11 | 0.08 |
| % 18:0 | 3.647 | 4.443 | 4.858 | 3.09 | 4.26 | 5.16 | 4.37 | 3.27 | 1.72 |
| % 18:1 n-9 | 60.213 | 62.184 | 49.853 | 56.41 | 51.10 | 49.73 | 0.00 | 0.11 | 3.30 |
| % 18:1 n-7 | 0.000 | 0.000 | 0.000 | 0.71 | 0.27 | 0.28 | 69.22 | 62.75 | 57.31 |
| % 18:2 | 11.097 | 13.890 | 12.594 | 7.11 | 8.57 | 9.89 | 5.24 | 6.74 | 19.80 |
| % 18:3 n-3 | 0.000 | 0.000 | 0.000 | 0.00 | 0.08 | 0.00 | 0.54 | 0.72 | 9.55 |
| % 20:0 | 1.723 | 1.031 | 1.137 | 1.78 | 2.20 | 2.52 | 0.85 | 0.49 | 0.60 |
| % 20:1 n-9 | 0.666 | 0.000 | 0.000 | 0.16 | 0.11 | 0.10 | 0.29 | 0.39 | 1.28 |
| % 22:0 | 1.368 | 1.291 | 1.316 | 1.22 | 1.75 | 1.86 | 0.69 | 0.59 | 0.34 |
| % 24:0 | 0.738 | 1.250 | 1.400 | 0.61 | 0.84 | 0.79 | 0.53 | 0.62 | 0.12 |
| % 16:0 + 18:0 | 18.9 | 12.47 | 10.74 | 18.76 | 20.42 | 25.39 | 19.78 | 24.72 | 6.22 |
| % Total Saturates | 24.052 | 21.004 | 31.264 | 23.06 | 26.2 | 31.52 | 22.27 | 27.68 | 7.56 |
| Cloud Point (° C.) | | | | 11.1 | 12.1 | 13 | 8.7 | 11 | −3.2 |
| Pour Point (° C.) | | | | 6 | 6 | 12 | 3 | 3 | −12 |
| CFPP (° C.) | | | | | | | 0 | 1 | −17 |

What is claimed is:

1. An oleaginous microorganism suitable for production of renewable materials, the microorganism comprising a genetic modification not present in an unmodified microorganism and a fatty acid methyl ester (FAME) profile that differs from the FAME profile of the unmodified microorganism when grown in culture.

2. The oleaginous microorganism of claim 1, wherein genetic engineering introduces the genetic modification.

3. The oleaginous microorganism of claim 1, wherein random mutagenesis introduces the genetic modification.

4. The oleaginous microorganism of claim 1, wherein the genetic modification alters the FAME profile of the modified microorganism.

5. The oleaginous microorganism of claim 1, wherein the genetic modification alters the production of one or more fatty acids.

6. The oleaginous microorganism of claim 1, wherein the genetic modification alters the biosynthetic pathway of fatty acids.

7. The oleaginous microorganism of claim 1, wherein the genetic modification alters one or more genes in the biosynthetic pathway of fatty acids.

8. The oleaginous microorganism of claim 1, wherein the genetic modification alters pyruvate dehydrogenase, acetyl-CoA carboxylase, acyl carrier protein, glycerol-3 phosphate acyltransferase, citrate synthase, stearoyl-ACP desaturase, glycerolipid desaturase, fatty acyl-ACP thioesterase, a fatty acyl-CoA reductase, a fatty aldehyde reductase, a fatty acyl-CoA aldehyde reductase, and/or a fatty aldehyde decarbonylase.

9. The oleaginous microorganism of any of claims 1-8, wherein the fermentation broth produced by the modified microorganism has a substantially similar cell density to the cell density of the fermentation broth produced by the unmodified microorganism.

10. The oleaginous microorganism of any of claims 1-8, wherein the culture of the modified microorganism comprises substantially similar conditions as the culture of the unmodified microorganism.

11. The oleaginous microorganism of any of claims 1-8, wherein each fermentation broth comprises a biomass of at least about 50 grams cellular dry weight per liter.

12. The oleaginous microorganism of any of claims 1-11, the microorganism being a yeast.

13. The oleaginous microorganism of claim 12, wherein the yeast belongs to the genus Rhodotorula, Pseudozyma, or Sporidiobolus.

14. The oleaginous microorganism of claim 13, the yeast being Sporidiobolus pararoseus, Pseudozyma rugulosa, Pseudozyma aphidis, or Rhodotorula ingeniosa.

15. The oleaginous microorganism of any of claims 1-14, the microorganism being the microorganism corresponding to one or more of ATCC Deposit No. PTA-13344 (Strain MK29404 Dry-1-321C) or ATCC Deposit No. PTA-13346 (Strain MK29404 248A).

16. The oleaginous microorganism of any of claims 1-14, the microorganism being the microorganism corresponding to one or more of ATCC Deposit No. PTA-13345 (Strain MK29794 30D) or ATCC Deposit No. PTA-13347 (Strain MK29794 117D).

17. The oleaginous microorganism of any of claims 1-14, the microorganism being the microorganism corresponding to one or more of ATCC Deposit No. PTA-13342 (Strain MK28428 8-500-3A) or ATCC Deposit No. PTA-13343 (Strain MK28428 149G).

18. The oleaginous modified microorganism of any of claims 1-17, the modified microorganism comprising a desirable FAME profile.

19. The oleaginous modified microorganism of any of claims 1-18, the modified microorganism comprising a more desirable FAME profile than the unmodified microorganism.

20. The oleaginous modified microorganism of any of claims 1-19, the modified microorganism comprising a more desirable FAME profile than another oleaginous organism.

21. The oleaginous modified microorganism of any of claims 1-20, the modified microorganism comprising a rapeseed-like FAME profile.

22. The oleaginous modified microorganism of any of claims 1-21, the modified microorganism comprising a FAME profile satisfying one or more specifications required by the biofuel standards of the United States, Canada, or European Union.

23. The oleaginous modified microorganism of any of claims 1-22, the modified microorganism comprising a FAME profile satisfying one or more specifications required by the biofuel standard EN14214.

24. The oleaginous modified microorganism of any of claims 1-22, the modified microorganism comprising a FAME profile satisfying one or more specifications according to current and/or subsequent revisions of EN14214.

25. The oleaginous modified microorganism of any of claims 1-24, the modified microorganism comprising a FAME profile satisfying one or more specifications required by the biofuel standard ASTM D6751.

26. The oleaginous modified microorganism of any of claims 1-25, the modified microorganism comprising a FAME profile satisfying one or more specifications according to current and/or subsequent revisions of ASTM D6751.

27. The oleaginous microorganism of any of claims 1-26, the FAME profile of the modified microorganism comprising a lower mass fraction (m/m) of total saturates than the FAME profile of the unmodified microorganism when grown in culture.

28. The oleaginous microorganism of claim 27, the FAME profile of the modified microorganism comprising a total saturates mass fraction (m/m) that is at least about 30 percent less than the FAME profile of the unmodified microorganism when grown in culture.

29. The oleaginous microorganism of any of claims 27-28, the FAME profile of the modified microorganism comprising a total saturates mass fraction (m/m) of about 19 percent or less when grown in culture.

30. The oleaginous microorganism of any of claims 27-29, the FAME profile of the modified microorganism comprising a total saturates mass fraction (m/m) of about 10 percent or less when grown in culture.

31. The oleaginous microorganism of any of claims 27-30, the FAME profile of the modified microorganism comprising a total saturates mass fraction (m/m) of about 5 percent or less when grown in culture.

32. The oleaginous microorganism of any of claims 27-31, the FAME profile of the modified microorganism comprising a total saturates mass fraction (m/m) of about 3 percent or less when grown in culture.

33. The oleaginous microorganism of any of claims 27-30, the FAME profile of the modified microorganism comprising a mass fraction (m/m) of total saturates between about 7 percent and about 10 percent when grown in culture.

34. The oleaginous microorganism of claim 27-30, the FAME profile of the modified microorganism comprising a total saturates mass fraction (m/m) of about 7.6 percent when grown in culture.

35. The oleaginous microorganism of any of claims 27-30, the FAME profile of the modified microorganism comprising a total saturates mass fraction (m/m) that is similar to the FAME profile of rapeseed oil.

36. The oleaginous microorganism of any of claims 1-35, the FAME profile of the modified microorganism comprising a lower mass fraction (m/m) of polyunsaturated methyl esters than the FAME profile of the unmodified microorganism when grown in culture.

37. The oleaginous microorganism of any of claims 1-36, the FAME profile of the modified microorganism comprising a polyunsaturated methyl ester mass fraction (m/m) of 1 percent or less.

38. The oleaginous microorganism of any of claims 1-37, the modified microorganism comprising a FAME profile comprising a lower mass fraction (m/m) of long chain saturated fatty acids than the FAME profile of the unmodified microorganism when grown in culture.

39. The oleaginous microorganism of any of claims 1-38, the FAME profile of the modified microorganism comprising a combined palmitic acid and stearic acid mass fraction (m/m) that is lower than the FAME profile of the unmodified microorganism when grown in culture.

40. The oleaginous microorganism of any of claims 1-39, the FAME profile of the modified microorganism comprising a combined palmitic acid and stearic acid mass fraction (m/m) of about 12.5 percent or less when grown in culture.

41. The oleaginous microorganism of claim 40, the FAME profile of the modified microorganism comprising a combined palmitic acid and stearic acid mass fraction (m/m) of about 10 percent or less when grown in culture.

42. The oleaginous microorganism of any of claims 40-41, the FAME profile of the modified microorganism comprising a combined palmitic acid and stearic acid mass fraction (m/m) of about 8 percent or less when grown in culture.

43. The oleaginous microorganism of any of claims 1-42, the FAME profile of the modified microorganism comprising a combined arachidic acid, behenic acid, and lignoceric acid mass fraction (m/m) of about 2 percent or less when grown in culture.

44. The oleaginous microorganism of any of claims 1-43, the FAME profile of the modified microorganism comprising a lower mass fraction (m/m) of palmitic acid than the FAME profile of the unmodified microorganism when grown in culture.

45. The oleaginous microorganism of claim 44, the FAME profile of the modified microorganism comprising a palmitic acid mass fraction (m/m) that is at least about 10 percent less than the FAME profile of the unmodified microorganism when grown in culture.

46. The oleaginous microorganism of any of claims 44-45, the FAME profile of the modified microorganism comprising a palmitic acid mass fraction (m/m) that is at least about 20 percent less than the FAME profile of the unmodified microorganism when grown in culture.

47. The oleaginous microorganism of any of claims 44-46, the FAME profile of the modified microorganism comprising a palmitic acid mass fraction (m/m) that is at least about 40 percent less than the FAME profile of the unmodified microorganism when grown in culture.

48. The oleaginous microorganism of any of claims 44-47, the FAME profile of the modified microorganism comprising a palmitic acid mass fraction (m/m) that is at least about 60 percent less than the FAME profile of the unmodified microorganism when grown in culture.

49. The oleaginous microorganism of any of claims 1-48, the FAME profile of the modified microorganism comprising a mass fraction (m/m) of palmitic acid that is about 10 percent or less when grown in culture.

50. The oleaginous microorganism of claim 49, the FAME profile of the modified microorganism comprising a mass fraction (m/m) of palmitic acid that is about 11 percent or less when grown in culture.

51. The oleaginous microorganism of any of claims 49-50, the FAME profile of the modified microorganism comprising a mass fraction (m/m) of palmitic acid that is about 15 percent or less when grown in culture.

52. The oleaginous microorganism of claim 49-51, the FAME profile of the modified microorganism comprising a mass fraction (m/m) of palmitic acid that is about 1.0 percent or more when grown in culture.

53. The oleaginous microorganism of any of claims 1-43, the FAME profile of the modified microorganism comprising a higher mass fraction (m/m) of palmitic acid than the FAME profile of the unmodified microorganism when grown in culture.

54. The oleaginous microorganism of any of claims 1-53, the FAME profile of the modified microorganism comprising a mass fraction (m/m) of palmitic acid that is between about 1.0 percent and about 10 percent when grown in culture.

55. The oleaginous microorganism of any of claims 1-54, the FAME profile of the modified microorganism comprising a lower mass fraction (m/m) of stearic acid than the FAME profile of the unmodified microorganism when grown in culture.

56. The oleaginous microorganism of claim 55, the FAME profile of the modified microorganism comprising a stearic acid mass fraction (m/m) that is at least about 10 percent less than the FAME profile of the unmodified microorganism when grown in culture.

57. The oleaginous microorganism of any of claims 55-56, the FAME profile of the modified microorganism comprising a mass fraction (m/m) of stearic acid that is about 2.5 percent or less when grown in culture.

58. The oleaginous microorganism of any of claims 55-57, the FAME profile of the modified microorganism comprising a mass fraction (m/m) of stearic acid that is about 0.5 percent or more when grown in culture.

59. The oleaginous microorganism of any of claims 55-58, the FAME profile of the modified microorganism comprising a mass fraction (m/m) of stearic acid that is between about 0.5 percent and about 2.5 percent when grown in culture.

60. The oleaginous microorganism of any of claims 1-54, the FAME profile of the modified microorganism comprising a higher mass fraction (m/m) of stearic acid than the FAME profile of the unmodified microorganism when grown in culture.

61. The oleaginous microorganism of any of claims 1-60, the FAME profile of the modified microorganism comprising a lower mass fraction (m/m) of myristic acid than the FAME profile of the unmodified microorganism when grown in culture.

62. The oleaginous microorganism of claim 61, the FAME profile of the modified microorganism comprising a myristic acid mass fraction (m/m) of about 1.5 percent or less when grown in culture.

63. The oleaginous microorganism of any of claims 61-62, the FAME profile of the modified microorganism comprising a myristic acid mass fraction (m/m) of about zero when grown in culture.

64. The oleaginous microorganism of any of claims 1-63, the FAME profile of the modified microorganism comprising a lower mass fraction (m/m) of arachidic acid than the FAME profile of the unmodified microorganism when grown in culture.

65. The oleaginous microorganism of claim 64, the FAME profile of the modified microorganism comprising a mass fraction (m/m) of arachidic acid that is about 1.5 percent or less when grown in culture.

66. The oleaginous microorganism of any of claims 64-65, the FAME profile of the modified microorganism comprising a mass fraction (m/m) of arachidic acid that is about zero when grown in culture.

67. The oleaginous microorganism of any of claims 1-66, the FAME profile of the modified microorganism comprising a lower mass fraction (m/m) of behenic acid than the FAME profile of the unmodified microorganism when grown in culture.

68. The oleaginous microorganism of claim 67, the FAME profile of the modified microorganism comprising a mass fraction (m/m) of behenic acid that is about 1.5 percent or less when grown in culture.

69. The oleaginous microorganism of any of claims 67-68, the FAME profile of the modified microorganism comprising a mass fraction (m/m) of behenic acid that is about zero when grown in culture.

70. The oleaginous microorganism of any of claims 1-69, the FAME profile of the modified microorganism comprising a lower mass fraction (m/m) of lignoceric acid than the FAME profile of the unmodified microorganism when grown in culture.

71. The oleaginous microorganism of claim 70, the FAME profile of the modified microorganism comprising a mass fraction (m/m) of lignoceric acid that is about 2.0 percent or less when grown in culture.

72. The oleaginous microorganism of any of claims 70-71, the FAME profile of the modified microorganism comprising a mass fraction (m/m) of lignoceric acid that is about 1.0 percent or less when grown in culture.

73. The oleaginous microorganism of any of claims 70-72, the FAME profile of the modified microorganism comprising a mass fraction (m/m) of lignoceric acid that is about 0.5 percent when grown in culture.

74. The oleaginous microorganism of any of claims 1-73, the FAME profile of the modified microorganism comprising a lower mass fraction (m/m) of palmitoleic acid than the FAME profile of the unmodified microorganism when grown in culture.

75. The oleaginous microorganism of claim 74, the FAME profile of the modified microorganism comprising a mass fraction (m/m) of palmitoleic acid that is about 1.0 percent or less when grown in culture.

76. The oleaginous microorganism of any of claims 74-75, the FAME profile of the modified microorganism comprising a mass fraction (m/m) of palmitoleic acid that is about 0.5 percent or less when grown in culture.

77. The oleaginous microorganism of any of claims 74-76, the FAME profile of the modified microorganism comprising a mass fraction (m/m) of palmitoleic acid that is about 0.4 percent when grown in culture.

78. The oleaginous microorganism of any of claims 1-77, the FAME profile of the modified microorganism comprising a lower mass fraction (m/m) of oleic acid than the FAME profile of the unmodified microorganism when grown in culture.

79. The oleaginous microorganism of any of claims 1-77, the FAME profile of the modified microorganism comprising a higher mass fraction (m/m) of oleic acid than the FAME profile of the unmodified microorganism when grown in culture.

80. The oleaginous microorganism of any of claims 1-79, the FAME profile of the modified microorganism comprising a mass fraction (m/m) of oleic acid that is about 70.0 percent or less when grown in culture.

81. The oleaginous microorganism of claim 80, the FAME profile of the modified microorganism comprising a mass fraction (m/m) of oleic acid that is about 50.0 percent or more when grown in culture.

82. The oleaginous microorganism of any of claims 80-81, the FAME profile of the modified microorganism comprising a mass fraction (m/m) of oleic acid that is between about 50.0 percent and about 70.0 percent when grown in culture.

83. The oleaginous microorganism of any of claims 1-82, the FAME profile of the modified microorganism comprising a lower mass fraction (m/m) of eicosenoic acid than the FAME profile of the unmodified microorganism when grown in culture.

84. The oleaginous microorganism of claim 83, the FAME profile of the modified microorganism comprising a mass fraction (m/m) of eicosenoic acid that is about 3.0 percent or less when grown in culture.

85. The oleaginous microorganism of any of claims 83-84, the FAME profile of the modified microorganism comprising a mass fraction (m/m) of eicosenoic acid that is about 1.0 percent or less when grown in culture.

86. The oleaginous microorganism of any of claims 83-85, the FAME profile of the modified microorganism comprising a mass fraction (m/m) of eicosenoic acid that is about 0.2 percent when grown in culture.

87. The oleaginous microorganism of any of claims 1-86, the FAME profile of the modified microorganism comprising a lower mass fraction (m/m) of erucic acid than the FAME profile of the unmodified microorganism when grown in culture.

88. The oleaginous microorganism of claim 87, the FAME profile of the modified microorganism comprising a mass fraction (m/m) of erucic acid that is about 5.0 percent or less when gown in culture.

89. The oleaginous microorganism of any of claims 87-88, the FAME profile of the modified microorganism comprising a mass fraction (m/m) of erucic acid that is about zero when grown in culture.

90. The oleaginous microorganism of any of claims 1-89, the FAME profile of the modified microorganism comprising a lower mass fraction (m/m) of linoleic acid than the FAME profile of the unmodified microorganism when grown in culture.

91. The oleaginous microorganism of any of claims 1-89, the FAME profile of the modified microorganism comprising a higher mass fraction (m/m) of linoleic acid than the FAME profile of the unmodified microorganism when grown in culture.

92. The oleaginous microorganism of any of claims 1-91, the FAME profile of the modified microorganism comprising a mass fraction (m/m) of linoleic acid that is about 35.0 percent or less when grown in culture.

93. The oleaginous microorganism of any of claims 1-92, the FAME profile of the modified microorganism comprising a mass fraction (m/m) of linoleic acid that is about 15.0 percent or more when grown in culture.

94. The oleaginous microorganism of any of claims 1-93, the FAME profile of the modified microorganism comprising a mass fraction (m/m) of linoleic acid that is between about 15.0 percent and about 35.0 percent when grown in culture.

95. The oleaginous microorganism of any of claims 1-94, the FAME profile of the modified microorganism comprising a lower mass fraction (m/m) of linolenic acid than the FAME profile of the unmodified microorganism when grown in culture.

96. The oleaginous microorganism of any of claims 1-94, the FAME profile of the modified microorganism comprising a higher mass fraction (m/m) of linolenic acid than the FAME profile of the unmodified microorganism when grown in culture.

97. The oleaginous microorganism of any of claims 1-96, the FAME profile of the modified microorganism comprising a mass fraction (m/m) of linolenic acid that is about 12.0 percent or less when grown in culture.

98. The oleaginous microorganism of any of claims 1-97, the FAME profile of the modified microorganism comprising a mass fraction (m/m) of linolenic acid that is about 6.0 percent or more when grown in culture.

99. The oleaginous microorganism of any of claims 1-97, the FAME profile of the modified microorganism comprising a mass fraction (m/m) of linolenic acid that is between about 6.0 percent and about 12.0 percent when grown in culture.

100. The oleaginous microorganism of claim 1, the FAME profile of the modified microorganism comprising:
a oleic acid mass fraction (m/m) of about 50 percent to about 70 percent;
a linoleic acid mass fraction (m/m) of about 15 percent to about 35 percent;
and
palmitic acid mass fraction (m/m) of less than about 10 percent.

101. The oleaginous microorganism of any of claims 1-100, the FAME profile of the modified microorganism comprising desirable cold flow properties.

102. The oleaginous microorganism of claim 101, the FAME profile of the modified microorganism comprising more desirable cold flow properties than the FAME profile of the unmodified microorganism when grown in culture.

103. The oleaginous microorganism of any of claims 101-102, the FAME profile of the modified microorganism comprising a cloud point less than about 4 degrees Celsius.

104. The oleaginous microorganism of any of claims 101-103, the FAME profile of the modified microorganism comprising a cloud point less than about −3 degrees Celsius.

105. The oleaginous microorganism of any of claims 101-104, the FAME profile of the modified microorganism comprising a pour point greater than about −18 degrees Celsius.

106. The oleaginous microorganism of any of claims 101-105, the FAME profile of the modified microorganism comprising a lower cloud point of than the FAME profile of the unmodified microorganism when grown in culture.

107. The oleaginous microorganism of any of claims 101-105, the FAME profile of the modified microorganism comprising a higher cloud point of than the FAME profile of the unmodified microorganism when grown in culture.

108. The oleaginous microorganism of any of claims 101-102, the FAME profile of the modified microorganism comprising a lower pour point of than the FAME profile of the unmodified microorganism when grown in culture.

109. The oleaginous microorganism of any of claims 101-102, the FAME profile of the modified microorganism comprising a higher pour point of than the FAME profile of the unmodified microorganism when grown in culture.

110. The oleaginous microorganism of any of claims 101-109, the FAME profile of the modified microorganism comprising a pour point less than about −9 degrees Celsius.

111. The oleaginous microorganism of any of claims 101-109, the FAME profile of the modified microorganism comprising a pour point between about −18 and about −9 degrees Celsius.

112. The oleaginous microorganism of any of claims 101-111, the FAME profile of the modified microorganism comprising a lower cold filter plugging point than the FAME profile of the unmodified microorganism when grown in culture.

113. The oleaginous microorganism of any of claims 101-112, the FAME profile of the OMM comprising a cold filter plugging point less than about zero degrees Celsius.

114. The oleaginous microorganism of any of claims 101-113, the FAME profile of the OMM comprising a cold filter plugging point less than about −5 degrees Celsius.

115. The oleaginous microorganism of any of claims 101-114, the FAME profile of the OMM comprising a cold filter plugging point less than −10 degrees Celsius.

116. The oleaginous microorganism of any of claims 1-115, wherein the modified microorganism produces a fermentation broth having a lower viscosity than a fermentation broth produced by the unmodified microorganism when grown in culture.

117. The oleaginous microorganism of any of claims 1-116, wherein the modified microorganism produces less exocellular polysaccharide than the unmodified microorganism.

118. A biofuel suitable for use in a compression engine, the biofuel comprising a fatty acid methyl ester (FAME) profile comprising:
a oleic acid mass fraction (m/m) of about 50 percent to about 70 percent;
a linoleic acid mass fraction (m/m) of about 15 percent to about 35 percent; and
a palmitic acid mass fraction (m/m) of less than about 10 percent;
wherein an oleaginous microorganism produces the biofuel.

119. A method of producing a biofuel precursor, the method comprising culturing the modified microorganism of any of claims 1-117 and collecting the fermentation broth produced by the microorganism.

120. A method of producing a biofuel, the method comprising:
(a) supplying a carbon source;
(b) converting the carbon source to fatty acids within the modified microorganism of any of claims 1-117;
(c) extracting fatty acids from the microorganism; and
(d) reacting the fatty acids to produce a biofuel.

121. The method of either claim 119 or 120, the modified microorganism being a yeast.

122. A biofuel precursor produced by the method of claim 121.

123. A biofuel derived from the biofuel precursor of claim 122.

124. A biofuel produced by the method of claim 120.

125. A method of powering a vehicle by combusting the biofuel of either claim 123 or 124 in an internal combustion engine.

126. A method for producing a renewable material, comprising growing the modified microorganism of any of claims 1-117 in a culture to produce a renewable material.

127. A renewable material produced by the method of claim 126.

128. A method for producing a biological oil, comprising growing the modified microorganism of any of claims 1-117 in a culture to produce a biological oil.

129. A method of producing a biological oil, the method comprising:
(a) supplying a carbon source;
(b) converting the carbon source to fatty acids within the modified microorganism of any of claims 1-117;
(c) extracting fatty acids from the microorganism; and
(d) reacting the fatty acids to produce a biological oil.

130. A biological oil produced by the method of either claim 128 or 129.

131. A composition produced by manufacturing the renewable material of claim 127.

132. The composition of claim 131, wherein the composition comprises food products, pharmaceutical compositions, cosmetics, or industrial compositions.

133. Use of the modified microorganism, fermentation broth, or culture of any of claims 1-117 for the manufacture of a renewable material.

134. Use of the modified microorganism, fermentation broth, or culture of any of claims 1-117 for the manufacture of a biofuel or biofuel precursor.

135. Use of the modified microorganism, fermentation broth, or culture of any of claims 1-117 for the manufacture of a food, supplement, cosmetic, or pharmaceutical composition for a non-human animal or human.
